(19) United States
(12) Patent Application Publication
(10) Pub. No.: US 2023/0390202 A1
(43) Pub. Date: Dec. 7, 2023

(54) CELLULAR MEMBRANE VESICLES AND USES THEREOF

(71) Applicant: University of Delaware, Newark, DE (US)

(72) Inventors: Eleftherios Papoutsakis, Newark, DE (US); Samik Das, Wilmington, DE (US)

(73) Assignee: University of Delaware, Newark, DE (US)

(21) Appl. No.: 18/032,094

(22) PCT Filed: Oct. 14, 2021
(86) PCT No.: PCT/US2021/054967

§ 371 Date: Apr. 14, 2023

Related U.S. Application Data
(60) Provisional application No. 63/092,544, filed on Oct. 16, 2020.

(51) Int. Cl. A61K 9/127; C12N 9/22

(52) U.S. Cl. CPC A61K 9/1272; C12N 9/22; C12N 2310/20

(57) ABSTRACT

The present invention provides a stable non-naturally occurring cellular membrane vesicle for delivering an active agent into a target cell. The cellular membrane vesicle comprises a biological membrane from a parent cell and a liquid medium encapsulated by the biological membrane. The liquid medium comprises an active agent and does not comprise a native cytoplasmic component of the parent cell. The biological membrane is specific for the target cell, and the active agent remains active upon delivery into the target cell. Also provided are methods for delivering the active agent with the cellular membrane vesicle and methods of preparing the cellular membrane vesicles.

### CROSS-REFERENCE TO RELATED APPLICATION

This application claims priority to U.S. Provisional Application No. 63/092,544, filed Oct. 16, 2020, and the contents of which are incorporated herein by reference in their entireties for all purposes.

### FIELD OF THE INVENTION

This invention relates generally to cellular membrane vesicles for delivery of active agents, for example, therapeutics and genome-editing molecules, to target cells by using biological membranes to encapsulate the active agents in a liquid medium.

### BACKGROUND OF THE INVENTION

Biological membranes such as cell membranes from primary mammalian cells or cultured mammalian cells from healthy individuals or patients have been used to wrap nanoparticles loaded by cargo. The nanoparticles are solid. The size of the resulting membrane wrapped nanoparticles is limited by the size of the nanoparticles, which usually have a diameter less than 200 nm, such that the loaded cargo is also limited in size. Extracellular vesicles (EVs) naturally produced by cells such as mammalian cells (e.g., exosomes) or bacteria (e.g., bacterial outer membrane vesicles (OMVs)), have also been used for targeted delivery of cargo. The size of the EVs is not tunable. Because the EVs are naturally produced by cells, the EVs carry native cytoplasmic components of the cells that may interfere with the biological activity of the cargo and cause undesirable effects on the target cells.

There remains a need for membrane vesicles having tunable size for carrying various active agents to target cells without interference from cytoplasmic components of the cells from which the membranes can be obtained and potentially used to generate the membrane vesicles.

### SUMMARY OF THE INVENTION

The present invention relates to novel stable non-naturally occurring cellular membrane vesicles for delivery of active agents into target cells and uses and preparation thereof.

A stable non-naturally occurring cellular membrane vesicle is provided for delivering an active agent into a target cell. The cellular membrane vesicle comprises a biological membrane from a parent cell and a liquid medium encapsulated by the biological membrane. The liquid medium comprises an active agent and does not comprise a native cytoplasmic component of the parent cell. The biological membrane is specific for the target cell. The active agent remains active upon delivery into the target cell. The cellular membrane vesicle may consist of the biological membrane and the liquid medium. The cellular membrane vesicle may have a diameter of 100-1000 nm.

The biological membrane may comprise a native surface receptor of the parent cell, and the native surface receptor may bind specifically to the target cell.

The parent cell may be selected from the group consisting of megakaryocytes (Mks), granulocytes, erythrocytes, platelets, monocytes, macrophages, lymphocytes, stem cells, endothelial cells, cardiac cells, bone cells, neuronal cells and tumor cells.

The target cell may be selected from the group consisting of hematopoietic stem & progenitor cells (HSPCs), adult stem cells, cardiac cells and neuronal cells.

In one embodiment of the cellular membrane vesicle, the parent cell may be a megakaryocyte (Mk) and the target cell may be a hematopoietic stem & progenitor cell (HSPC).

The active agent may be selected from the group consisting of proteins, nucleoproteins, nucleic acids, organic molecules, and combinations thereof.

The target cell may express a guide RNA (gRNA) specific for a target native gene of the target cell, and the active agent may comprise Cas9 that binds specifically to the gRNA. The active agent may further comprise a therapeutic.

The active agent may comprise a ribonucleoprotein (RNP) of Cas9 and a guide RNA (gRNA) specific for a target native gene of the target cell. The active agent may further comprise a therapeutic.

The liquid medium may further comprise a soluble polymer. The soluble polymer may be selected from the group consisting of polycation polyethyleneimine (PEI), non-toxic polycations, polyanionic polymers, and nonionic triblock copolymers. For example, the soluble polymer may be polycation polyethyleneimine (PEI).

A method for delivery of an active agent into a target cell is also provided. The delivery method comprises contacting the stable non-naturally occurring cellular membrane vesicle with the target cell, and releasing the active agent into the target cell from the cellular membrane vesicle. The active agent remains active upon release into the target cell.

According to the delivery method, the active agent may be released into the target cell within 120 minutes after the contacting step.

The delivery method may further comprise fusing the biological membrane with a cytoplasmic membrane of the target cell after the contacting step and before the releasing step.

According to the delivery method, the target cell may express a guide RNA (gRNA) specific for a target native gene of the target cell, and the active agent may comprise Cas9 that binds specifically to the gRNA. The delivery method may further comprise editing of the target native gene in the target cell.

According to the delivery method, the active agent may comprise a ribonucleoprotein (RNP) of Cas9 and a guide RNA (gRNA) specific for a target native gene of the target cell. The delivery method may further comprise editing of the target native gene in the target cell.

In one embodiment of the delivery method, the parent cell may be a megakaryocyte (Mk), the target cell may be a hematopoietic stem & progenitor cell (HSPC), and the active agent may comprise a ribonucleoprotein (RNP) of Cas9 and a guide RNA (gRNA) specific for a target native gene in the HSPC. The delivery method may further comprise editing the target native gene in the target cell.

In another embodiment of the delivery method, the parent cell may be a megakaryocyte (Mk), the target cell may be a hematopoietic stem & progenitor cell (HSPC) expressing a guide RNA (gRNA) specific for a target native gene of the target cell, and the active agent may comprise Cas9 that binds specifically to the gRNA. The delivery method may further comprise editing the target native gene in the target cell.

According to the delivery method, the liquid medium may further comprise a soluble polymer. The soluble polymer may be selected from the group consisting of polycation polyethyleneimine (PEI), non-toxic polycations, polyanionic polymers, and nonionic triblock copolymers. For example, the soluble polymer may be polycation polyethyleneimine (PEI).

For each stable non-naturally occurring cellular membrane vesicle of the present invention, a preparation method is provided. The preparation method comprises isolating the biological membrane from the parent cell, and encapsulating the liquid medium by the biological membrane. The liquid medium comprises the active agent. As a result, the stable non-naturally occurring cellular membrane vesicle is prepared.

The preparation method may further comprise mixing the isolated biological membrane and the biological liquid medium, and the liquid medium may further comprise a soluble polymer such that a mixture may be obtained. The preparation method may further comprise extruding the mixture through an extruder pore. The soluble polymer may be selected from the group consisting of polycation polyethyleneimine (PEI), non-toxic polycations, polyanionic polymers, and nonionic triblock copolymers. For example, the soluble polymer may be polycation polyethyleneimine (PEI). The isolated biological membrane and the biological liquid medium may be mixed at a weight ratio from 10:1 to 1:10. The extruder pore may have a diameter from 1 nm to 1000 nm.

### BRIEF DESCRIPTION OF THE DRAWINGS

FIG. 1 shows that vesicles from cell-derived membranes may facilitate targeted cargo delivery to various cells such as hematopoietic stem and progenitor cells (HSPCs). Various cargo molecules in a liquid solution or liquid suspension may be wrapped with natural cell membranes to create a vector for targeted cargo delivery. According to one embodiment of the present invention, vesicles from megakaryocytic (Mk) cells derived from HSPCs may be generated and loaded in order to develop targeted delivery to HSPCs. These membrane-wrapped therapeutics may facilitate cell-specific cargo delivery to HSPCs both in vitro and in vivo for direct correction of cellular defects. Different cell membranes with target specificity may be used to generate vesicles for targeted delivery of cargo molecules to other cell types and organs in the human and mammal body.

FIG. 2 shows that megakaryocytic and megakaryocyte-like membrane vesicles may encapsulate CRISPR Cas9-associated cargo for cell-specific delivery and gene therapy of hematopoietic stem and progenitor cells (HSPCs). Gene-specific CRISPR Cas9 nucleoprotein, discrete Cas9 and single-guided RNA (sgRNA), or Cas9 plasmids may be effectively encapsulated within megakaryocytic and/or other cell membranes for in vitro or in vivo delivery to HSPCs and other target cells. Membrane-wrapped Cas9 and the associated sgRNA(s) improves gene editing efficiency, specificity and safety over traditional, plasmid-based Cas9 treatments. Membrane wrapping imparts cell specificity and bioavailability of the Cas9 therapeutic through targeted delivery to the specified cells. Delivery of the Cas9 or similar genome editing proteins and the corresponding nucleoprotein complexes of Cas9 and guide RNA molecules assures safety compared to delivery of plasmid DNA encoding for the Cas9 and guide RNA molecules.

FIG. 3 shows that co-delivery of a guide RNA (gRNA) plasmid with Cas9 nuclease improves gene knockout over plasmid-based systems. (A) Percentage of CD41⁺ cells in 1) untreated CHRF cells (control) and cells after electroporation with: 2) plasmid for expression of a Cas9-GFP fusion protein of Cas9 and green fluorescent protein (GFP) and CD41 sgRNA (CHRF+Cas9-GFP_gRNA v2), 3) Cas9-GFP nuclease and plasmid expressing sgRNA targeting the CD41 gene, and a BFP (blue) fluorescent-protein tag (CHRF+BFP_gRNA v2 Cas9(n)) and 4) Cas9-GFP nuclease and plasmid expressing sgRNA targeting the CD41 gene, and a mTq2 (turquoise) fluorescent tag (CHRF+mTq2_gRNA v2 Cas9(n)). (B) Viability of CHRF cells after each Cas9, sgRNA delivery method as described in 3A determined via TO-PO-3 (far red) viability staining. No significant differences in viability were found between any of the Cas9-sgRNA methods. (C) Presence of Cas9-GFP from each Cas9-GFP delivery method. Direct electroporation of Cas9-GFP nuclease led to significant increase in the proportion of cells containing Cas9-GFP, including a sustained presence Cas9-GFP in ≈40% of cells 72 hours after electroporation.

FIG. 4 shows that providing two targets for sgRNA significantly increases Cas9-mediated gene knockout efficiency in CHRF cells with no significant impact to viability. (A) Percentage of CD41⁺ cells in 1) untreated CHRF cells (control) and cells after electroporation with: 2) plasmid expressing Cas9-GFP and CD41 sgRNA (that is sgRNA targeting the CD41 gene) (CHRF+Cas9-GFP_gRNA v2), 3) Cas9-GFP nuclease and plasmid expressing CD41 sgRNA and a mTq2 (turquoise) fluorescent tag for one locus (CHRF+mTq2_gRNA v2 Cas9(n)) and 4) Cas9-GFP nuclease with two mTq2-tagged plasmids expressing CD41 sgRNA for different loci each (CHRF+mTq2_2TgRNA v2 Cas9(n)). Over 60% of CHRF cells containing sgRNA for two gene loci exhibited disruption of CD41 expression after 24 hours as measured via flow cytometry as compared to <25% for one sgRNA gene locus and <5% for the combined Cas9-sgRNA plasmid alone. (B) Viability of CHRF cells after each Cas9, sgRNA delivery method (as described in 4A) as determined via TO-PO-3 (far red) viability staining. No significant differences in viability were found between any of the Cas9-sgRNA methods.

FIG. 5 shows that different combinations of membrane vesicle (MV)-wrapped forms of Cas9, sgRNA allow for high flexibility and customization of various Cas9-mediated gene therapies. Cellular MVs here means vesicles using cellular membranes and carrying a liquid cargo free from cytoplasmic cellular material. Multiple combinations of Cas9 in either a (purified) nuclease form (Cas9 nuclease only), as a nucleoprotein complex with the associated sgRNA (Cas9-sgRNA ribonucleoprotein), or as a plasmid may be delivered to HSPCs for gene therapy (sgRNA-expressing plasmid DNA). For sgRNA, discrete cellular MVs may solely contain a plasmid expressing for a specific sgRNA, or a “hybrid” loaded cellular MV may be produced by extruding purified Cas9 and the sgRNA plasmid together. Using these discrete combinations enable the implementation of different sgRNA gene targets by simply introducing cellular MVs containing the associated sgRNA plasmid sequence(s), and thus, increase the scope and simplicity of Cas9 gene therapy.

FIG. 6 shows that highly branched polyethyleneimine (PEI 25-kDa and PEI 750-kDa) can complex Cas9-GFP and CHRF^{PMA} membranes to facilitate efficient Cas9 delivery to CHRF cells and HSPCs through co-incubation. (A) Cas9-GFP loaded CHRF membrane vesicles (MVs) were prepared by premixing and extruding 8 ng/mL solution of Cas9-GFP with PKH26-stained PMA-treated CHRF membranes together with different soluble polymers (0.01% PEI 25-kDa (25K), 0.01% PEI 750-kDa (750K), and 0.01% Poloxamer F68; wt/wt %). The fraction of incubated CD34⁺ HSPCs cells containing Cas9-GFP was measured through flow cytometry. (B) Cas9-GFP loaded CHRF cellular MVs were prepared with various concentrations (wt/wt %) of PEI 25K and PEI 750K, and the fraction of Cas9-GFP+ CHRF cells was measured via flow cytometry. (C, D): CHRF cellular MVs carrying Cas9 were prepared with 0.01% (C) or (D) 0.005% PEI 750K, and the Cas9-loaded cellular MVs were incubated with CHRF calls which were screened for Cas9-GFP uptake. CHRF cells fixed and stained at 68-72 hrs were imaged with confocal microscopy, with Cas9-GFP represented as the bright spots in and around the cell and the phalloidin-AF647 stained actin cytoskeleton represented as light grey. The PKH26-stained CHRF cellular MVs and DAPI-stained nucleus was also imaged (not highlighted). Cellular MVs here means vesicles using cellular membranes and carrying a liquid cargo free from cytoplasmic cellular material.

FIG. 7 shows that centrifugal filtration of Cas9-CHRF^{PMA}-PEI 2-kDa, 25-kDa complex reduces free solution PEI-induced cytotoxicity while retaining cellular MV-wrapped Cas9. After preparation of PEI 2-kDa and 25-kDa-encapsulated Cas9-loaded CHRF cellular MVs via extrusion, these Cas9-PEI cellular MVs were subsequently purified via ultrafiltration with 100-kDa centrifugal filters. A) CHRF cells incubated with ultrafiltered Cas9-PEI cellular MVs prepared with 0.01% PEI 25-kDa had substantially higher viability than unfiltered Cas9-PEI cellular MVs as determined via flow cytometry (3 biological replicates). B) Significant amounts of free PEI 2-kDa and PEI 25-kDa were recovered from the filter flowthrough, and C) subsequent NTA analysis indicated that the wrapped Cas9-PEI cellular MVs were successfully retained in the filter retentate. D) Purified Cas9-PEI CHRF cellular MVs prepared with PEI 2-kDa yielded significantly higher cell viability than Cas9-PEI cellular MVs prepared with PEI 25-kDa across a broad range of PEI concentrations. Cellular MVs here means vesicles using cellular membranes and carrying a liquid cargo free from cytoplasmic cellular material. *: p<0.05, **: p<0.01; student's T-test.

FIG. 8 shows that, in one embodiment, a composition of the Cas9-PEI cellular MVs is tailored for CHRF cells and HSPCs to maximize Cas9 uptake and minimize cytotoxicity. After determining PEI 2-kDa as the optimal PEI material for Cas9 encapsulation and membrane wrapping, A) CHRF cells and B) HSPCs were incubated with Cas9-PEI CHRF cellular MVs (CHRF cells), or Cas9-PEI MkMVs (HSPCs) prepared with a range of different PEI 2-kDa concentrations and tested for viability via flow cytometry. To determine the level of Cas9-GFP uptake with the range of prepared Cas9-PEI cellular MVs, C) CHRF cells and D) HSPCs were screened for presence of GFP fluorescence via flow cytometry. All conditions were tested using 3 biological replicates. Cellular MVs here means vesicles using cellular membranes and carrying a liquid cargo free from cytoplasmic cellular material.

FIG. 9 shows that purified Cas9-GFP-PEI 2-kDa cellular MVs facilitate effective Cas9 delivery to both hematopoietic stem and progenitor cells (HSPCs) and CHRF-288 cells. Cas9-GFP loaded Mk- and CHRF-membrane cellular MVs were prepared with either 0.75% (MkMVs) or 0.05% (CHRF cellular MVs) PEI 2-kDa and purified via ultrafiltration. A) HSPCs and B) CHRF cells were incubated with Cas9-PEI MkMVs and Cas9-PEI CHRF cellular MVs, respectively, for 24 hours and seeded and fixed onto poly-L-lysine coated coverslips. The seeded cells were subsequently stained with phalloidin and DAPI to visualize the actin cytoskeleton and nucleus. Both A) HSPCs and B) CHRF cells displayed substantial uptake of the membrane-wrapped (PKH26) Cas9-GFP (circled on Cas9-GFP channel images) Cellular MVs into the cell as shown by extensive Cas9-GFP and cellular MV fluorescence within the periphery of actin-stained cytoskeleton. Most of the imaged HSPCs contained some degree of Cas9-GFP fluorescence as indicated by the wide view images (top panels of A and B), and confirmation of Cas9-uptake was determined via Cas9-fGFP fluorescence inside the cell in the magnified images (bottom panels of A and B). Cellular MVs here means vesicles using cellular membranes and carrying a liquid cargo free from cytoplasmic cellular material. Scale bars: 20-μm for wide view images and 5-μm in magnified view images.

FIG. 10 shows that cellular MV-delivered Cas9-sgRNA nucleoprotein yields gradual uptake and effective gene disruption in HSPCs following co-incubation. Prior to assessing CD34 gene disruption in HSPCs, Cas9-GFP-sgCD34 ribonucleoproteins (RNPs) were wrapped with D12 Mk membranes and prepared and purified as previously described. A) Incubation of HSPCs with Cas9-sgCD34-PEI cellular MVs yields gradual uptake of the Cas9 RNP over time, thus demonstrating the applicability of the Cas9-PEI cellular MV system to facilitate controlled Cas9 delivery and Cas9-mediated gene therapy. B) Cellular MV-delivered Cas9-sgCD34 yielded robust disruption of the CD34 gene with fewer than 40% of Cas9+HSPCs expressing the gene in comparison to >65% of HSPCs directly electroporated with the RNP. C) Flow cytometry contour plots showing steady increase of CD34-negative HSPC population across all conditions. HSPCs incubated with Cas9-sgCD34-PEI cellular MVs exhibited greatest increase in CD34-negative population after 24-hours when compared to Cas9 plasmid DNA and Cas9 RNP electroporation conditions. Cellular MVs here means vesicles using cellular membranes and carrying a liquid cargo free from cytoplasmic cellular material. *: p<0.05, **: p<0.01; student's T-test.

FIG. 11 shows that gene therapy of HSPCs provides many benefit via hematopoietic differentiation. (A) Treating and correcting genetic disorders in HSPCs leads to different lineages of healthy blood cells as the HSPCs differentiate. This requires a smaller effective therapeutic dose, as fewer overall cells need to be treated to produce a healthy population of cells. (B) Natural moieties and features on the outside of the Mk membrane vesicles permit HSPC-specific interaction with no or reduced interaction with other cell types.

FIG. 12 shows that, in one embodiment, Mk membrane-wrapped therapeutics using the disclosed cellular MVs target HSPCs in vivo. Wrapping Cas9 and/or other cargo in Mk membranes facilitates localization of the cargo-loaded MkMVs to the bone marrow and other tissues where HSPCs reside. This allows for tissue-specific gene therapy without the need for ex vivo treatment and infusion. Cellular MVs here means vesicles using cellular membranes and carrying a liquid cargo free from cytoplasmic cellular material.

FIG. 13 shows a graphical overview of preparation of cellular membrane vesicles (MVs) according to one embodiment of the invention. Cellular membrane vesicle source, delivered cargo, and polymer encapsulate may be modified to treat a broad variety of cells and diseases. Using different polymer active agents (e.g., proteins, nucleic acids, and small molecule drugs) may be wrapped with a range of different natural cell membranes. This allows targeted delivery of active agents to a variety of cells along with tunable release of the active agents via selection of different polymers. Cellular MVs here means vesicles using cellular membranes and carrying a liquid cargo free from cytoplasmic cellular material.

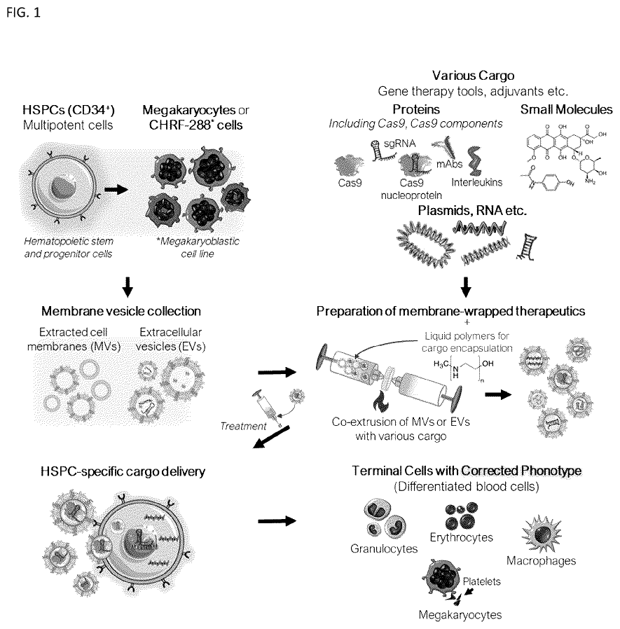

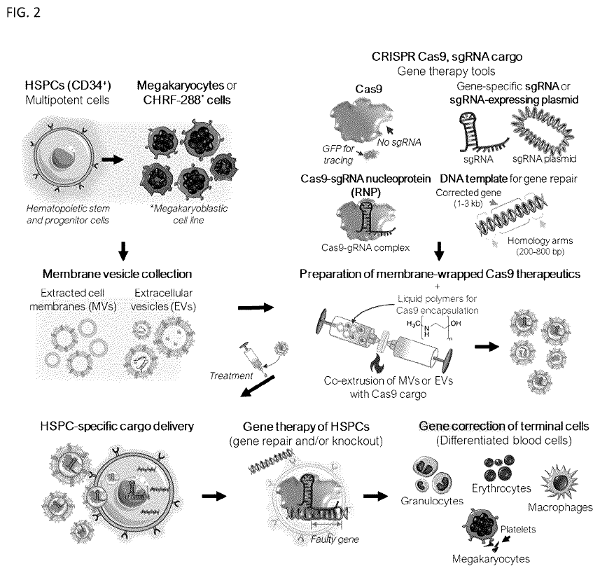

### DETAILED DESCRIPTION OF THE INVENTION

The present invention provides novel cellular membrane vesicles (MVs) each comprising a biological membrane from a parent cell and an active agent in a liquid medium encapsulated by the biological membrane, and uses of these cellular membrane vesicles for delivering the active agent to a target cell. Recognition and cargo delivery is a complex process that requires machinery beyond that of the membrane properties alone, such as accessory proteins and nucleic acids native to megakaryocytic microparticles (MkMPs). The inventors have surprisingly discovered that natural cell membranes from megakaryocytes (Mk) are sufficient for targeted cargo delivery specifically to homotypic and hematopoietic stem and progenitor cell (HSPC). This discovery makes it possible to use cell membranes from other human donors, Mk cells developed from primary laboratory cultures, or Mk-like cell lines.

The inventors have successfully isolated Mk membranes mechanically and loaded them with non-native liquid cargo, including but not limited to CRISPR Cas9 ribonucleoprotein for delivery to HSPCs for genome engineering and cell/gene therapy. The encapsulation of the Cas9 ribonucleoprotein within isolated Mk or Mk-like cell membranes was accomplished via extrusion. This MkMV-encapsulated Cas9 ribonucleoprotein complex brings the versatility of targeted Cas9 gene therapy to ameliorate a variety of genetic hematological disorders, which have been historically difficult to remediate. This invention enables utilization of the CRISPR Cas9 system to produce precise gene-specific edits. The inventors have showcased the versatility of the decoupled Cas9 nuclease and sgRNA system by demonstrating its improvement in Cas9-mediated gene editing efficiency over traditional, plasmid-based systems as exhibited in other studies along with its predicted wide scope and scalability. The inventors have demonstrated delivering Cas9 and sgRNA as either discretely-loaded cellular MVs or combined within the same cellular MV, which increases the scope of treatments without the need to completely redesign the system for each treatment. Additionally, the natural Mk membrane vesicles are natively targeting homotypic or HSPCs cells with high specificity, and thus do not require additional protein engineering to impart cell for targeted cargo delivery to the desirable cell type. Finally, the inventors have demonstrated the tunability of the membrane-encapsulated Cas9 system to provide robust delivery and uptake of Cas9 and Cas9 ribonucleoproteins for a variety of different types of cells. Thus, by coupling the HSPC targeting ability of the Mk membrane with the precision of Cas9-guided gene editing, the inventors have greatly improved the simplicity and efficiency for gene therapy of HSPCs. Additionally, targeted cell therapies are expected to yield increased Cas9 bioavailability in vivo, which should subsequently translate to smaller doses of Cas9 to remain effective.

This invention provides immense potential for treating a broad spectrum of genetic hematological disorders, as the repaired HSPCs differentiate and expand into mature blood cells with the corrected phenotype. This approach, namely of creating cellular MVs from, for example, cell membranes that are loaded with desirable liquid cargo, may be applied to delivery of many proteins, nucleoproteins, nucleic acids and various organic drugs. It may also be used to encapsulate for targeted delivery of whole organelles and even encapsulated microbes for various therapeutic applications. Beyond Mk membranes, membranes from other cell types may be used to target different cell types and organs in the body.

The inventors have used Mk or Mk-like membrane-vesicles to encapsulate drug and other molecules including proteins (such as Cas9), ribonucleoproteins, and nucleic acids, for targeted delivery of the encapsulated cargo to desirable target cells. The Mk membrane may provide prolonged circulation in vivo thus decreasing the potential of immunogenicity while improving the bioavailability of the cargo to be delivered to target cells, including HSPCs.

The inventors have showed that delivering the Cas9-based editing machinery as a ribonucleoprotein rather than through a DNA vector provides great control of the level of Cas9 in the cells would avoid random integration of a Cas9-expressing plasmid. With this approach, gene-specific sgRNA for multiple discrete targets may be delivered simultaneously with Cas9 and limit the variability from issues involving expression of both Cas9 and sgRNA from a single vector. This may also reduce the probability of any off-target gene editing effects of Cas9 arising from overexpression of Cas9 in vivo via a vector.

The inventors have also demonstrated that protein solutions, Cas9, and nucleic acids together with liquid cationic polymers, such as polyethyleneimine may be successfully wrapped with cellular membranes for targeted delivery to specific cells. The formulation of the membrane-encapsulated Cas9 ribonucleoprotein-loaded vesicles may also be tuned to improve compatibility and delivery efficiency for specific types of cells.

The inventors have further showcased that cellular MV-delivered Cas9 ribonucleoprotein yields more robust gene editing of HSPCs than direct delivery (e.g., electroporation). This shows that the cellular MV system may provide improved functionality and efficacy of the Cas9 ribonucleoprotein upon delivery to HSPCs or other target cells.

The term “cellular membrane vesicle” as used herein refers to an artificial, non-naturally occurring, particle comprising a space enclosed by a biological membrane and filled with a liquid medium. The biological membrane encapsulates the liquid medium. The liquid medium is in the space enclosed by the biological membrane, and is not in the biological membrane. For example, in the examples below, a cellular MV means a vesicle formed from a cellular membrane and carrying a liquid, aqueous solution cargo free from a cytoplasmic component or cellular material.

The term “encapsulate,” “encapsulated” or “encapsulation” as used herein refers to wrapping, coating or covering a liquid medium or components therein by a biological membrane. The liquid medium is in the space enclosed by the biological membrane, and is not in the biological membrane.

The term “stable” or “stability” as used herein refers to the ability of a substance (e.g., an active agent or cellular membrane vesicle) to remain substantially biologically active or substantially intact, without substantial degradation or deterioration, over a predetermined time under predetermined conditions. The substance may maintain at least 80%, 85%, 90%, 95% or 99% of its original biological activity, weight or size after storage for a predetermined time, for example, for at least 1, 2, 3, 4, 5, 6 or 7 days, 1, 2, 3 or 4 weeks, or 1, 2, 3, 6 or 12 months, at a temperature of, for example, 25-40° C. or room temperature.

The term “specific for” as used herein refers to the ability of a substance, for example, a particle, cellular membrane vesicle (MV) or a portion thereof (e.g., a biological membrane of the cellular MV), vector, a nucleic acid (e.g., a guide RNA (gRNA)), or a protein (e.g., antibody), to recognize a desirable target, for example, a target cell (e.g., a HSPC), nucleic acid (e.g., a target gene) or antigen with specificity of, for example, at least about 60%, 65%, 70%, 75%, 80%, 85%, 90%, 95% or 99%, as compared with a non-target control. The recognition of the targeted cells may be mediated through a biological molecule (e.g., a surface receptor) on the surface of the particle, cellular MV or vector, or a biological structure (e.g., a biological membrane of the cellular MV) capable of recognizing the target cell. The recognition may cause the particle, cellular MV or vector to move towards the target cell, and optionally bind specifically to the target cell, directly or indirectly, via a covalent or non-covalent bond or interaction.

The term “native” as used herein refers to the cellular source of a biological component. A biological component in or from a cell is native to the cell. The biological component may be in or from the cytoplasm, nucleus or cell membrane of the cell. The biological component may be a biological molecule, for example, a protein, a nucleic acid or a combination thereof.

The terms “isolating” and “purifying” as used herein are interchangeably and refer to separating a component (e.g., a biological molecule or a component of a cell) from other components in a structure (e.g., a mixture or cell). The isolated or purified component has a higher concentration or purity after the separation as compared with that before the separation.

The present invention provides a stable non-naturally occurring cellular membrane vesicle for delivering an active agent into a target cell. The cellular membrane vesicle comprises a biological membrane and a liquid medium. The liquid medium is encapsulated by the biological membrane. The liquid medium comprises an active agent and does not comprise a native cytoplasmic component of the parent cell. The biological membrane is from a parent cell and is specific for the target cell. The active agent remains active upon delivery into the target cell. In one embodiment, the cellular membrane vesicle consists of the biological membrane and the liquid medium. The term “native cytoplasmic component” refers to any material contained within the cytoplasm of a cell, that is the material contained within the space defined by the cytoplasmic membrane of the cell. Those would include but are not limited to any protein, nucleic acid, lipid, their precursors and derivatives, and metabolic intermediates.

The biological membrane may be a membrane derived from a native membrane of the parent cell, for example, a native cytoplasmic membrane or a native membrane of an organelle (e.g., nucleus, endoplasmic reticulum, Golgi apparatus, mitochondria, and plastids) of the parent cell, or a combination thereof. For example, the biological membrane is from a native cytoplasmic membrane of a parent cell. The biological membrane may comprise a phospholipid bilayer. The parent cell may be a mammalian, plant, yeast, prokaryote or insect cell, for example, a human cell. In one embodiment, the parent cell is a human cell and the biological membrane may be a cytoplasmic membrane of the human cell.

The liquid medium may be a medium in the form of a liquid. The liquid medium may be an aqueous solution containing soluble solutes and/or colloidal particles in an aqueous solvent. The liquid medium may be substantially free of a solid or semisolid substance, allowing for encapsulation or solvation of variably-sized active agents. The liquid medium may comprise one or more solid substances at a concentration less than about 10, 9, 8, 7, 6, 5, 4, 3, 2, 1, 0.5, 0.1, 0.05, 0.01, 0.005 or 0.001 wt % based on the total weight of the liquid medium. The liquid medium may have a viscosity below the viscosity that characterizes an amorphous-solid gel at temperatures above 20° C. Typical viscosities of liquid media range from 0.5 to less than 20 centipoise (cP), while gel viscosities would range from 200 to over 30,000 cP. One cP=0.01 Poise (P). One poise=0.1 Pascal second.

The liquid medium may further comprise a soluble polymer. The soluble polymer soluble in the liquid medium. The soluble polymer may be selected from the group consisting of polycation polyethyleneimine (PEI), non-toxic polycations, polyanionic polymers, and nonionic triblock copolymers, also known as poloxamers. In one embodiment, the soluble polymer may be polycation polyethyleneimine (PEI).

The cellular membrane vesicle may have a tunable size. The size of the cellular membrane vesicles may be adjusted to a predetermined size by modifying the volume of the liquid medium. The cellular membrane vesicle may have a diameter of at least about 100, 200, 300, 400 or 500 nm, no more than 500, 600, 700, 800, 900 or 1000 nm, or in the range of about 100-1000, 200-1000, 300-1000, 400-1000, 500-1000, 100-500, 200-500, 300-500 or 400-500 nm.

The parent cell may be any cell having a biological membrane. The parent cell may be a primary cell or a cultured cell. The cell may be a mammalian, plant, yeast, prokaryote, bacterial or insect cell, which may be human cell. The human cell may be from a healthy individual or a patient. The parent cell may be a cancer cell.

The parent cell may be selected from the group consisting of megakaryocytes (Mks), granulocytes, erythrocytes, platelets, monocytes, macrophages, lymphocytes, stem cells, endothelial cells, cardiac cells, bone cells, neuronal cells and tumor cells. The granulocytes include neutrophils, eosinophils and basophils. The lymphocytes include T cells and B cells. The stem cells include mesenchymal stem cells and endothelial progenitor cells.

The target cell may be a cell in or from a subject, or a cultured cell. The subject may be a mammal, for example, a human. The subject may be a healthy individual or a patient. The patient may be in need of the active agent. The target cell may be a cancer cell. The target cell may be selected from the group consisting of hematopoietic stem & progenitor cells (HSPCs), adult stem cells, cardiac cells and neuronal cells.

The biological membrane may comprise a native membrane protein of the parent cell. The native membrane protein may be a native surface receptor of the parent cell. The surface receptor may bind specifically to the target cell. Examples of the surface receptors include but are not limited to CD34, which is the character cell surface marker for HSPCs, CD11b, a surface marker common to neutrophils and macrophages.

In one embodiment, the parent cell is a megakaryocyte (Mk) and the target cell is a hematopoietic stem & progenitor cell (HSPC). MKs are large bone marrow cells with a lobated nucleus responsible for production of blood thrombocytes (i.e., platelets). The MKs are derived from a HSPC in the bone marrow. The MKs may be CD41⁺, CD42b⁺ and/or CD61⁺. The HSPCs are present in or isolated from blood and bone marrow and capable of forming mature blood cells, for example, red blood cells, platelets or white blood cells. The HSPCs may be CD34⁺, CD34⁺, CD59⁺, CD90/Thy1⁺, CD38^{low/-}, c-Kit^{-/low}, and/or Lin⁻.

The active agent may be any substance having a biological activity. The active agent may be a biological molecule, an organic molecule or a combination thereof. The active agent may be a therapeutic or diagnostic agent. The active agent remains stable in the cellular membrane vesicle and/or during delivery to the target cells. The active agent may maintain at least 80%, 85%, 90%, 95% or 99% of its original activity or weight after storage in the cellular membrane vesicle for a predetermined time, for example, for at least 1, 2, 3, 4, 5, 6 or 7 days, 1, 2, 3 or 4 weeks, or 1, 2, 3, 6 or 12 months, at a temperature of, for example, 25-40° C. or room temperature.

The active agent may be selected from the group consisting of proteins, nucleoproteins, nucleic acids, organic molecules, small-molecule drugs and combinations thereof. The protein may be an antibody, interleukin, or gene-editing agent. The gene-editing protein may be selected from the group consisting of CRISPR protein (e.g., Cas9, Cas12 or one of Cas 13 proteins such as Cas 13a, 13b, 13c and 13d), transcription activator-like effector nucleases (TALEN), zinc-finger nucleases (ZFNs), meganucleases and nickases. The nucleoproteins may be ribonucleoproteins (RNPs), for example, a RNP of Cas9 and sgRNA (Cas9-sgRNA). The nucleic acids include small RNAs (e.g., siRNAs and miRNAs) and large RNAs, linear and plasmid DNA, and genes. The organic molecules may be chemotherapeutic agents.

In one embodiment, the target cell may express a guide RNA (gRNA) specific for a target native gene of the target cell, and the active agent may comprise Cas9 that binds specifically to the gRNA. The active agent may further comprise a therapeutic. The therapeutic may be an adjuvant or a nucleic acid.

In another embodiment, the active agent may comprise a ribonucleoprotein (RNP) of Cas9 and a guide RNA (gRNA) specific for a target native gene of the target cell. The active agent may further comprise a therapeutic. The therapeutic may be an adjuvant or a nucleic acid.

For each of the cellular membrane vesicle of the present invention, a method for delivery of an active agent into a target cell. The cellular membrane vesicle comprises the biological membrane and the liquid medium. The liquid medium is encapsulated by the biological membrane. The liquid medium comprises an active agent and does not comprise a native cytoplasmic component of the parent cell. The biological membrane is from a parent cell and is specific for the target cell. The delivery method comprises contacting the stable cellular membrane vesicle with the target cell, and releasing the active agent into the target cell from the cellular membrane vesicle. The active agent remains active upon release into the target cell. In one embodiment, the cellular membrane vesicle consists of the biological membrane and the liquid medium.

According to the delivery method, the biological membrane may be a membrane derived from a native membrane of the parent cell, for example, a native cytoplasmic membrane or a native membrane of an organelle (e.g., nucleus, endoplasmic reticulum, Golgi apparatus, mitochondria, and plastids) of the parent cell, or a combination thereof. For example, the biological membrane is from a native cytoplasmic membrane of a parent cell. The biological membrane may comprise a phospholipid bilayer. The parent cell may be a mammalian, plant, yeast, prokaryote or insect cell, for example, a human cell. In one embodiment, the parent cell is a human cell and the biological membrane may be a cytoplasmic membrane of the human cell.

According to the delivery method, the liquid medium may be a medium in the form of a liquid. The liquid medium may be an aqueous solution containing soluble solutes and/or colloidal particles in an aqueous solvent. The liquid medium may be substantially free of a solid or semisolid substance. The liquid medium may comprise one or more solid substances at a concentration less than about 10, 9, 8, 7, 6, 5, 4, 3, 2, 1, 0.5, 0.1, 0.05, 0.01, 0.005 or 0.001 wt % based on the total weight of the liquid medium. The liquid medium may be of low viscosity (0.5 to 20 cP), thus allowing for encapsulation or solvation of active agents of variable size. The active agent may range in size from small molecules to 100 nm. The liquid medium may further comprise a soluble polymer. The soluble polymer may be used up to its maximum solubility in the aqueous liquid medium. The soluble polymer may be selected from the group consisting of polycation polyethyleneimine (PEI), non-toxic polycations, polyanionic polymers, and nonionic triblock copolymers, also known as poloxamers. In one embodiment, the soluble polymer may be polycation polyethyleneimine (PEI).

According to the delivery method, the cellular membrane vesicle may have a tunable size. The size of the cellular membrane vesicles may be adjusted to a predetermined size by modifying the volume of the liquid medium. The cellular membrane vesicle may have a diameter of at least about 100, 200, 300, 400 or 500 nm, no more than 500, 600, 700, 800, 900 or 1000 nm, or in the range of about 100-1000, 200-1000, 300-1000, 400-1000, 500-1000, 100-500, 200-500, 300-500 or 400-500 nm.

According to the delivery method, the parent cell may be any cell having a biological membrane. The parent cell may be a primary cell or a cultured cell. The cell may be a mammalian, plant, yeast, prokaryote, bacterial or insect cell, for example, a human cell. The human cell may be from a healthy individual or a patient. The parent cell may be a cancer cell. The parent cell may be selected from the group consisting of megakaryocytes (Mks), granulocytes, erythrocytes, including erythrocytes, platelets, monocytes, macrophages, lymphocytes, stem cells, endothelial cells, cardiac cells, bone cells, neuronal cells, fibroblasts or fibroblastoid cells, iPS cells, hepatic, mesenchymal stem cells, fat and tumor cells. The granulocytes include neutrophils, eosinophils and basophils. The lymphocytes include T cells and B cells. The stem cells include mesenchymal stem cells and endothelial progenitor cells.

According to the delivery method, the target cell may be a cell in or from a subject, or a cultured cell. The subject may be a mammal, for example, a human. The subject may be a healthy individual or a patient. The patient may be in need of the active agent. The target cell may be a cancer cell. The target cell may be selected from the group consisting of hematopoietic stem & progenitor cells (HSPCs), adult stem cells, cardiac cells, neuronal and brain cells, and tumor cells.

According to the delivery method, the active agent may be released into the target cell immediately after the contacting step. For example, the active agent may be released into the target cell within about 10, 20, 30, 40, 60, 80, 100 or 120 minutes after the cellular membrane vesicle is in contact with the target cell. At least about 50, 60, 70, 80, 90, 95 or 99 wt % of the active agent in the cellular membrane vesicle may be released from the cellular membrane vesicle into the target cell. The active agent may be any substance having a biological activity. The active agent may be a biological molecule, an organic molecule or a combination thereof. The active agent may be a therapeutic or diagnostic agent. The active agent remains stable in the cellular membrane vesicle and/or during delivery to the target cells. The active agent may maintain at least 80%, 85%, 90%, 95% or 99% of its original weight after storage in the cellular membrane vesicle for a predetermined time, for example, for at least 1, 2, 3, 4, 5, 6 or 7 days, 1, 2, 3 or 4 weeks, or 1, 2, 3, 6 or 12 months, at a temperature of, for example, 25-40° C. or room temperature. The suitable active agent may be selected from the group consisting of proteins, nucleoproteins, nucleic acids, organic molecules, small molecule drugs, and combinations thereof. The protein may be an antibody, interleukin, or gene-editing agent. The gene-editing protein may be selected from the group consisting of CRISPR protein (e.g., Cas9, Cas12 or one of Cas 13 proteins such as Cas 13a, 13b, 13c and 13d), transcription activator-like effector nucleases (TALEN), zinc-finger nucleases (ZFNs), meganucleases and nickases. The nucleoproteins may be ribonucleoproteins (RNPs), for example, a RNP of Cas9 and sgRNA (Cas9-sgRNA). The nucleic acids include small RNAs (e.g., siRNAs and miRNAs) and large RNAs, linear and plasmid DNA, genes encoding the proteins. The organic molecules may be chemotherapeutic agents.

The delivery method may further comprise fusing the biological membrane with a cytoplasmic membrane of the target cell after the contacting step and before the releasing step. The biological membrane may comprise a native surface receptor of the parent cell, and the surface receptor may bind specifically to the target cell. Examples of the surface receptors include CD34, which is the character cell surface marker for HSPCs, CD11b, a surface marker common to neutrophils and macrophages.

In one embodiment of the delivery method, the target cell may express a guide RNA (gRNA) specific for a target native gene of the target cell, and the active agent may comprise Cas9 that binds specifically to the gRNA. The delivery method may further comprise editing of the target native gene in the target cell.

In another embodiment of the delivery method, the active agent may comprise a ribonucleoprotein (RNP) of Cas9 and a guide RNA (gRNA) specific for a target native gene of the target cell. The delivery method may further comprise editing of the target native gene in the target cell.

In yet another embodiment of the delivery method, the parent cell may be a megakaryocyte (Mk), the target cell may be a hematopoietic stem & progenitor cell (HSPC), and the active agent may comprise a ribonucleoprotein (RNP) of Cas9 and a guide RNA (gRNA) specific for a target native gene in the HSPC. The delivery method may further comprise editing of the target native gene in the HSPC.

In yet another embodiment of the delivery method, the parent cell may be a megakaryocyte (Mk), the target cell may be a hematopoietic stem & progenitor cell (HSPC) expressing a guide RNA (gRNA) specific for a target native gene of the HSPC, and the active agent may comprise Cas9 that binds specifically to the gRNA. The delivery method may further comprise editing of the target native gene in the HSPC.

For each stable cellular membrane vesicle of the present invention, the invention further provides a method of preparing the stable cellular membrane vesicle. The preparation method comprises isolating a biological membrane from a parent cell, and encapsulating a liquid medium by the biological membrane. As a result, a stable cellular membrane vesicle is prepared. The cellular membrane vesicle comprises the biological membrane and the liquid medium. The liquid medium is encapsulated by the biological membrane. The liquid medium comprises an active agent and does not comprise a native cytoplasmic component of the parent cell. The biological membrane is from the parent cell and is specific for the target cell. The active agent remains active upon delivery into the target cell. In one embodiment, the cellular membrane vesicle consists of the biological membrane and the liquid medium.

According to the preparation method, the biological membrane may be a membrane derived from a native membrane of the parent cell, for example, a native cytoplasmic membrane or a native membrane of an organelle (e.g., nucleus, endoplasmic reticulum, Golgi apparatus, mitochondria, and plastids) of the parent cell, or a combination thereof. For example, the biological membrane is from a native cytoplasmic membrane of a parent cell. The biological membrane may comprise a phospholipid bilayer. The parent cell may be a mammalian, plant, yeast, prokaryote or insect cell, for example, a human cell. In one embodiment, the parent cell is a human cell and the biological membrane may be a cytoplasmic membrane of the human cell.

According to the preparation method, the liquid medium may be an aqueous solution containing soluble solutes and/or colloidal particles in an aqueous solvent. The liquid medium may be substantially free of a solid or semisolid substance. The liquid medium may comprise one or more solid substances at a concentration less than about 10, 9, 8, 7, 6, 5, 4, 3, 2, 1, 0.5, 0.1, 0.05, 0.01, 0.005 or 0.001 wt % based on the total weight of the liquid medium. The liquid medium may have a viscosity less than 20 cP.

Where the liquid medium further comprises a soluble polymer, the preparation method may further comprise mixing the isolated biological membrane and the biological liquid medium so that a mixture is obtained, and then extruding the mixture through an extruder pore. As a result, the stable cellular membrane vesicle is prepared. The soluble polymer may be used up to its maximum solubility in the aqueous liquid medium. The soluble polymer may be selected from the group consisting of polycation polyethyleneimine (PEI), non-toxic polycations, polyanionic polymers, and nonionic triblock copolymers, also known as poloxamers. In one embodiment, the soluble polymer may be polycation polyethyleneimine (PEI).

The isolated biological membrane and the biological liquid medium may be mixed at a weight ratio from about 20:1 to about 1:20, 10:1 to about 1:10, from about 5:1 to about 1:5, or from about 2:1 to about 1:2. The extruder pore may have a diameter from about 1 nm to about 1000 nm, from about 100 nm to about 1000 nm, from about 200 nm to about 1000 nm, from about 500 nm to about 1000 nm, from about 1 nm to about 500 nm, from about 100 nm to about 500 nm, or from about 200 nm to about 500 nm.

According to the preparation method, the prepared cellular membrane vesicle may have a tunable size. The size of the cellular membrane vesicles may be adjusted to a predetermined size by modifying the volume of the liquid medium. The cellular membrane vesicle may have a diameter of at least about 100, 200, 300, 400 or 500 nm, no more than 500, 600, 700, 800, 900 or 1000 nm, or in the range of about 100-1000, 200-1000, 300-1000, 400-1000, 500-1000, 100-500, 200-500, 300-500 or 400-500 nm.

According to the preparation method, the parent cell may be any cell having a biological membrane. The parent cell may be a primary cell or a cultured cell. The cell may be a mammalian, plant, yeast, prokaryote, bacterial or insect cell, for example, a human cell. The human cell may be from a healthy individual or a patient. The parent cell may be a cancer cell. The parent cell may be selected from the group consisting of megakaryocytes (Mks), granulocytes, erythrocytes, platelets, monocytes, macrophages, lymphocytes, stem cells, endothelial cells, cardiac cells, bone cells, neuronal cells and tumor cells. The granulocytes include neutrophils, eosinophils and basophils. The lymphocytes include T cells and B cells. The stem cells include mesenchymal stem cells and endothelial progenitor cells. The biological membrane may comprise a native membrane protein of the parent cell. The native membrane protein may be a native surface receptor of the parent cell. The surface receptor may bind specifically to the target cell. Examples of the surface receptors include CD34, which is the character cell surface marker for HSPCs, CD11b, a surface marker common to neutrophils and macrophages.

According to the preparation method, the target cell may be a cell in or from a subject, or a cultured cell. The subject may be a mammal, for example, a human. The subject may be a healthy individual or a patient. The patient may be in need of the active agent. The target cell may be a cancer cell. The target cell may be selected from the group consisting of hematopoietic stem & progenitor cells (HSPCs), adult stem cells, cardiac cells and neuronal cells.

In one embodiment of the preparation method, the parent cell may be a megakaryocyte (Mk) and the target cell may be a hematopoietic stem & progenitor cell (HSPC).

According to the preparation method, the active agent may be any substance having a biological activity. The active agent may be a biological molecule, an organic molecule or a combination thereof. The active agent may be a therapeutic or diagnostic agent. The active agent remains stable in the cellular membrane vesicle and/or during delivery to the target cells. The active agent may maintain at least 80%, 85%, 90%, 95% or 99% of its original weight after storage in the cellular membrane vesicle for a predetermined time, for example, for at least 1, 2, 3, 4, 5, 6 or 7 days, 1, 2, 3 or 4 weeks, or 1, 2, 3, 6 or 12 months, at a temperature of, for example, 25-40° C. or room temperature. The active agent may be selected from the group consisting of proteins, nucleoproteins, nucleic acids, organic molecules, small molecule drugs, and combinations thereof. The protein may be an antibody, interleukin, or gene-editing agent. The gene-editing protein may be selected from the group consisting of CRISPR protein (e.g., Cas9, Cas12 or one of Cas 13 proteins such as Cas 13a, 13b, 13c and 13d), transcription activator-like effector nucleases (TALEN), zinc-finger nucleases (ZFNs), meganucleases and nickases. The nucleoproteins may be ribonucleoproteins (RNPs), for example, a RNP of Cas9 and sgRNA (Cas9-sgRNA). The nucleic acids include small RNAs (e.g., siRNAs and miRNAs) and large RNAs, linear and plasmid DNA, genes encoding the proteins. The organic molecules may be chemotherapeutic agents.

In one embodiment of the preparation method, the target cell may express a guide RNA (gRNA) specific for a target native gene of the target cell, and the active agent may comprise Cas9 that binds specifically to the gRNA. The active agent may further comprise a therapeutic. The therapeutic may be an adjuvant or a nucleic acid.

In another embodiment of the preparation method, the active agent may comprise a ribonucleoprotein (RNP) of Cas9 and a guide RNA (gRNA) specific for a target native gene of the target cell. The active agent may further comprise a therapeutic. The therapeutic may be an adjuvant or a nucleic acid.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### Example 1. Cellular Membrane Vesicles (MVs)

Megakaryocytic (Mk) membranes were isolated mechanically and loaded with non-native cargo, including but not limited to CRISPR Cas9 nucleoprotein for delivery to hematopoietic stem and progenitor cells (HSPCs) for genome engineering and cell/gene therapy.

Materials. All reagents and basal media were obtained from Millipore Sigma and Thermo Fisher unless otherwise indicated. Purified Cas9-GFP nuclease and sgRNA constructs for specific genes in both gRNA and plasmid DNA form were purchased from GenScript. Primary human CD34⁺ HSPCs were obtained from the Fred Hutchinson Cancer Research Center (Washington). All interleukins required to induce megakaryopoiesis from HSPCs (rhSCF, rhTPO, IL-3, IL-6, IL-9, and IL-11) were purchased from PeproTech. The cellular MV extrusion instruments (syringes, heating block, Teflon membrane supports, filters etc.) were purchased from Avanti Polar Lipids, and Whatman® polycarbonate membrane inserts were purchased from GE Lifesciences. All fluorescently conjugated antibodies for staining and measurement via flow cytometry were purchased from BD Lifesciences. MACS magnetic columns and magnetically-conjugated beads for CD61⁺ cell selection and dead cell exclusion were purchased from Miltenyi Biotec.

Developing and maintaining primary Mk cultures and CHRF-288-11 cell line. Day 12 (D12) megakaryocytes were developed from primary CD34 cells (hematopoietic stem and progenitor cells; HSPCs). Cells were cultured in IMDM base media supplemented with BIT 9500 serum substitute, and cells were passaged in media with different cytokine cocktails on Day 5 and Day 7. CHRF-288-11 cells were prepared in IMDM base media supplemented with 10% fetal bovine serum and were passaged every 3-4 days. To induce a megakaryocytic phenotype, the cells were treated with 1 ng of phorbol 12-myrsitate 13-6 acetate (PMA) per mL of media for 3 days. The PMA-treated CHRF (CHRF^{PMA}) cells, which become adherent after treatment, were dissociated with Accutase and harvested for membrane collection. Day 0-5 CD34+ cells were incubated at 37° C. with 5% CO₂, 5% O₂, and 95% relative humidity, while Day 5-12 CD34+/CD61+/Mk cells and CHRF cells were incubated at 37° C. with 5% CO₂, 20% O₂, and 85% relative humidity.

Transfection of CHRF cells with Cas9 variants. Before assessing the production and delivery efficiency of polymer-encapsulated Cas9, the efficacy of Cas9-mediated gene therapy in CHRF cells was tested by directly transfecting the cells with Cas9 and Cas9 components. For these experiments, approximately 2 μg of purified Cas9-GFP and 8 μg of plasmid DNA for either Cas9-anti CD41 sgRNA or fluorescently-tagged anti CD41 sgRNA were added to an electroporation cuvette containing 2 million CHRF cells resuspended in 100 μL of electroporation buffer (Lonza Kit V). The cells were electroporated using the T-003 protocol on the Lonza Nucleofector II, and the freshly electroporated cells were immediately quenched with 400 μL of warmed CHRF media prior to transfer to 2 mL of warmed media (each) in a 6 well plate. To remove traces of the electroporation buffer, the transfected cells were washed and resuspended in fresh CHRF media 24 hours after electroporation.

Assessing gene knockout efficiency in transfected CHRF cells. After successful transfection of the CHRF cells, each cell was tested for expression of CD41, a surface marker characteristic of megakaryocytes, and for viability. At each assessed timepoint following electroporation (24 hrs, 48 hrs, and 72 hrs), 50 μL of the CHRF cell suspension was mixed with 5 μL of PE anti-CD41 antibodies (BD Lifesciences) and 50 μL of 1 μM TO-PO-3 (viability stain), and the stained cells were incubated for 15 mins at 4° C. to minimize nonspecific antibody binding. After cold incubation, 200 μL of filtered 1×PBS was added to each sample, which were subsequently measured for fluorescence via flow cytometry (BD FACSAria II). Presence of Cas9, expressed sgRNA, CD41, and viability were measured and gated under their respective fluorescence channels against both unstained and untreated controls.

Producing Mk and CHRF^{PMA} membrane vesicles. Following cell maturation and activation, Mk and CHRF^{PMA} cells were harvested, the cell pellet was washed in 1×PBS and stained with PKH26, an orange/red lipophilic membrane dye. After quenching the dye with 1% BSA, the stained cells were washed again in 1×PBS and lysed and suspended in a hypotonic lysis buffer (20 mM Tris HCl, 10 mM KCl, 2 mM MgCl₂) and protease inhibitor (p8340, Sigma), and subsequently homogenized in a Dounce homogenizer with both coarse and tight pestles. The nuclear pellet was removed from the lysate after centrifugation at 3,200×g for 5 mins at 4° C., and the mitochondrial pellet was sequentially removed after ultracentrifugation at 20,000×g for 20 mins at 4° C. Finally, the membranes were pelleted and collected after ultracentrifugation at 100,000×g for 50 mins at 4° C., and the resulting membrane pellet was resuspended in sterile biology-grade water. Vesicle concentrations were determined via Nanoparticle Tracking Analysis (NTA; NanoSight NS300) prior to co-extrusion with the desired cargo. Note: many of the examples showcased in this disclosure use CHRF^{PMA} membranes to demonstrate the proof of concept, but each example can be effectively adapted to an Mk membrane-only system.

Polymer encapsulation of Cas9-GFP solutions. To help facilitate optimal membrane wrapping of Cas9-GFP in solution, the Cas9 solution was premixed with positively-charged polyethyleneimine to increase the zeta potential of the negatively-charged Cas9 nuclease prior to wrapping with the negatively-charged cell membranes. For this process PEI-encapsulated Cas9-GFP, PEI 25-kDa (lower PEI branch weight) and 750-kDa (higher PEI branch weight) was coated onto purified Cas9-GFP by mixing 3 μg of purified Cas9-GFP with various wt./wt. % aqueous polymer solutions (0.05%, 0.01%, and 0.005%). After a short incubation at 25° C., the Cas9 premix was extruded through a 400-nm polycarbonate membrane at 55° C. for 11 times via the Avanti lipid extruder (more procedural details in next section). Finally, the concentration of PEI-coated Cas9 was determined via NTA, and confirmation of PEI coating was confirmed by measuring zeta potential before and after PEI coating (Anton Paar LiteSizer 500).

Producing Mk and CHRF^{PMA} membrane-wrapped gene therapeutics. Prior to wrapping the Cas9-GFP solutions, a concentration ratio of 2:1 membrane:polymer-protein (as determined via NTA) were premixed with a variable volume of sterile biology-grade water to yield a total volume of 700 μL. Subsequently, the premix was added to a 1 mL syringe and was warmed to 55° C. via the extruder heating block from the Avanti lipid extruder. Next, a 400-nm pore polycarbonate membrane and filters were applied between two Teflon supports and affixed to the extruder heating block, along with an empty 1 mL syringe opposite to the MV-polymer-protein premix-filled syringe. Finally, the premix was passed 11 times across the 400-nm membrane, ensuring that the final pass transfers the extruded sample into the originally empty syringe to avoid any contamination with unextruded protein and membranes.

Purification of Cas9-PEI (2-kDa) Mk and CHRF^{PMA} membrane vesicles. Following extrusion of the Cas9-PEI cellular MVs, the extruded samples were cooled to −4° C. prior to purification. Next, the extruded Cas9-PEI cellular MV suspension was added to a 100-kDa PES centrifugal filter (Sartorius), and the loaded centrifugal filters were centrifuged at 2,000×g for 10 minutes. After centrifugation, the purified Cas9-PEI cellular MV sample was decanted from the top chamber of the centrifugal filter insert, and the concentration and particle size distribution was determined via NTA (NanoSight NS300). To determine the amount of free PEI in the centrifugal filter flowthrough, the concentration of PEI was measured using a colorimetric assay.

Co-incubation with cargo-loaded cellular MVs for assessment of cargo delivery. After sufficient quantities of both wrapped and unwrapped Cas9-GFP-laden cellular MVs were produced, both wrapped and unwrapped polymer-protein sample was split between replicates and were incubated with 500,000 CHRF cells suspended in 50 μL of CHRF media in a 1.5 mL Eppendorf tube at 37° C. for 2 hours. After this initial co-incubation, the contents of each tube were transferred to a 24 well plate containing 700 μL of pre-warmed CHRF media per well. The cells were finally incubated at 37° C. with 5% CO₂, 20% O₂, and 85% relative humidity, and 50 μL of each cell suspension was pulled at different timepoints (6, 24, 48, and 72 hours) for analysis via flow cytometry. For these analyses, 50 μL of the cell suspension was mixed with 1 μM propidium iodide (PI) and incubated for 15 minutes at RT for viability staining, and the cells were measured for GFP/FITC and viability. Each of these experiments also included untreated controls and conditions with direct incubation of equivalent volumes (to amount in loaded cellular MVs) of the purified Cas9-GFP nuclease solution.

Assessing gene knockout efficiency in HSPCs following co-incubation with Cas9-GFP ribonucleoprotein loaded cellular MVs. Prior to extrusion with Mk MVs, Cas9-GFP ribonucleoproteins were prepared by incubating Cas9-GFP with pooled sgRNA corresponding to different targets within the CD34 gene, a characteristic surface marker of primitive undifferentiated HSPCs. The prepared Cas9-sgCD34 ribonucleoproteins (RNPs) were subsequently encapsulated with PEI 2-kDa, wrapped with Mk membranes, and purified as previously described; an equivalent amount of the Cas9-sgCD34 RNP was reserved for direct transfection of HSPCs. Next, equivalent numbers of HSPCs preconditioned with base media (80% IMDM, 20% BIT 9500, and stem cell factor (SCF)) were either incubated with the Cas9-sgCD34 RNP-loaded MkMVs or electroporated with Cas9-GFP-sgCD34-expressing plasmid DNA (8 μg/1M cell ratio) or the equivalent amount of unwrapped Cas9-sgCD34 RNP. At each assessed timepoint following electroporation (6 hrs, 24 hrs, 48 hrs, and 72 hrs), 50 μL of the HSPC cell suspension was mixed with 5 μL of PE anti-CD34 antibodies (BD Lifesciences) and 50 μL of 1 μM TO-PO-3 (viability stain), and the stained cells were incubated for 15 mins at 4° C. to minimize nonspecific antibody binding. After cold incubation, 200 μL of filtered 1×PBS was added to each sample, which were subsequently measured for fluorescence via flow cytometry (BD FACSAria II). Presence of Cas9 uptake, CD34, and viability were measured and gated under their respective fluorescence channels against both unstained and untreated controls.

Imaging CHRF cells and HSPCs after co-incubation with Cas9-GFP loaded cellular MVs. CHRF cells and HSPCs from different incubation timepoints were seeded onto poly-L-lysine coated coverslips and were subsequently fixed with 4% paraformaldehyde (PFA) in PBS. Following a few washed with filtered PBS, the seeded coverslips were coated and stained with 200 μl of 0.0025% Alexa Fluor 647-conjugated phalloidin at RT for 30 minutes; the phalloidin staining was used to help visualize the actin cytoskeleton and morphology of the fixed cells. Finally, the stained coverslips were washed with filtered PBS, mounted onto microscope slides with SlowFade with DAPI (Invitrogen), sealed and stored at 4° C. until analysis.

Statistical analysis. Significance was determined via paired Student's T-test, and is denoted as *p<0.05, **p<0.01 unless stated otherwise. Error bars are shown as ±standard error of the mean (SEM).

Example 1A. Cas9-mediated gene editing is more robust through the co-delivery of Cas9 nuclease with a sgRNA-expressing plasmid than the traditional method of delivering a vector with combined expression of Cas9-gRNA.

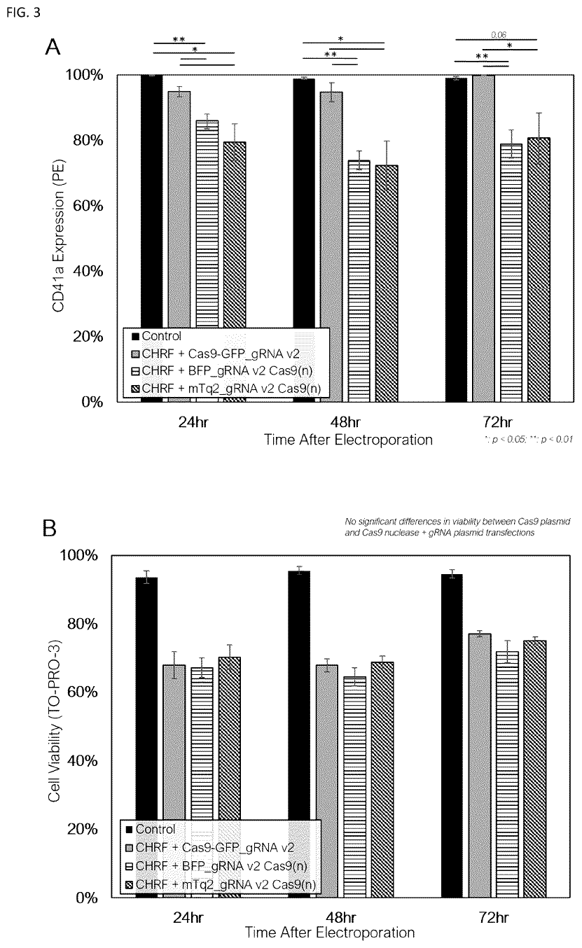

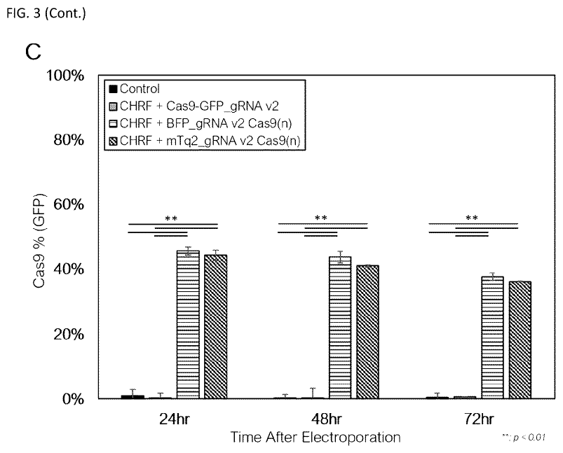

CRISPR Cas9-mediated gene therapy has rightfully dominated scientific and medical literatures alike, primarily due to the simplicity and overall scope of providing precise, sequence-specific edits to a gene. Many current applications for Cas9-based therapy involve delivering a plasmid to cells which may express both Cas9 and the gene-associated sgRNA, but issues with limited expression and insertional mutagenesis curb overall gene editing efficiency. Here, we show that directly electroporation CHRF cells with the Cas9-GFP purified nuclease and a plasmid expressing CD41-specific sgRNA results in CD41 gene knockout, with over 25% of CHRF cells losing expression of CD41 after 48 hours, in contrast to only 5% of cells transfected with the combined Cas-sgRNA plasmid (FIG. 3A). Moreover, there was no significant difference in the viability of the cells between each of the Cas9 delivery approaches (FIG. 3B). Finally, direct delivery of the Cas9 nuclease further sustained the presence of Cas9 intracellularly, with >33% of cells containing Cas9-GFP after 72 hours (FIG. 3C), which could result in increased gene editing efficiency.

Example 18. Pooling Cas9 nucleases with sgRNA vectors with 2 distinct targets provides efficient, multiple gene editing of cells in vitro without any negative impart to overall cell viability.

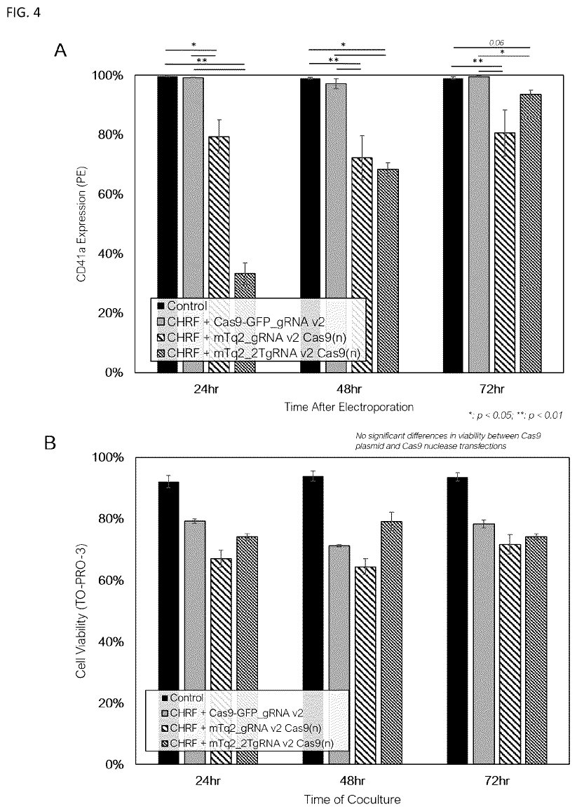

We next tested the overall impact on gene editing efficiency if the number of gene targets for Cas9-mediated knockout were increased from one to two. The overall amount of Cas9 was kept the same as in Example 1A, but two distinct sgRNA-expressing plasmids were delivered simultaneously with each sgRNA-expressing plasmid corresponding to different target sequences within the CD41 gene. As expected, the dual sgRNA-expressing plasmid delivery provided significant improvement over the plasmid-only Cas9 transfection (FIG. 4A), but over 66% of the CHRF cells measured were no longer CD41+24 hours post transfection, as opposed to 21% and 1% for the single sgRNA target and the Cas9-sgRNA combined plasmid, respectively. This demonstrates the versatility of our system, as multiple gene targets can be handled simultaneously without sacrificing much efficacy. With this approach, complex, multi-gene disorders can be treated simultaneously. Importantly, the dual sgRNA-expressing plasmid delivery had insignificant impact to the overall cell viability when compared to the single sgRNA-expressing and Cas9-expressing plasmid transfection (FIG. 4B), therefore coupling improved gene editing efficiency without any negative impact to cell health.

Example 1C. Splitting delivery of sgRNA vectors and Cas9 nucleases as both Cas9-loaded cellular membrane vesicles (MVs) and discrete sgRNA-expressing plasmid-loaded cellular MVs provides efficient and targeted gene editing along with the added flexibility of multiple gene targets due to multiple combinations of different sgRNA-expressing plasmids via cellular MVs.

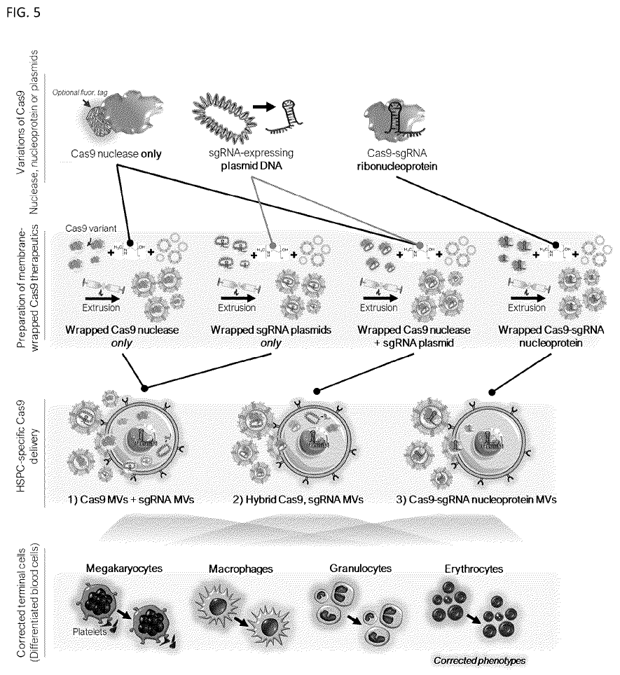

As mentioned previously, one of the hallmarks of CRISPR Cas9-based gene therapy is its simplicity and wide scope of gene targets facilitated by just modifying the sequences of the associated sgRNA. However, many proposed vector-based systems avoid combining discrete sgRNA gene targets into the same and/or multiple vectors, thus curtailing the scope of multiple Cas9-mediated gene therapies simultaneously. As shown in previous examples, decoupling the Cas9 nuclease from its complementary, gene-specific sgRNA provides robust gene editing, but it also provides other auxiliary benefits outside of improved gene editing efficiency. As shown in FIG. 5, multiple variants of Cas9 and sgRNA can be delivered to cells via cellular MVs: 1) simultaneous delivery of distinct Cas9 nuclease-loaded and sgRNA-expressing plasmid-loaded cellular MVs, 2) “hybrid” cellular MV loaded with both Cas9 and an associated sgRNA-expressing plasmid vector, and 3) cellular MVs containing the Cas9-sgRNA precomplexed ribonucleoprotein. As the cellular membrane vesicles can be obtained from a variety of different cells, different genes in different cells can be modulated, as the tropism of each loaded cellular MV is set by the characteristics of the outer membrane. From a biopharmaceutical perspective, having discrete Cas9-loaded and sgRNA-expressing plasmid-loaded cellular MVs improves the scalability of the overall system, as Cas9-loaded cellular MVs may be produced en masse while sgRNA-expressing plasmid-laden cellular MVs may be individually produced as per the patient's needs; cellular MVs can also be generated from a variety of tissues sources from either autologous or allogeneic donors. This also permits tunability of the effectiveness of each treatment: a low gene editing efficiency may be adjusted by increasing the dose of either the Cas9-loaded cellular MVs or sgRNA-expressing plasmid-loaded cellular MVs, and thus, the nature of each treatment may be tailored to the biology of the treated individual.

Example 1D. The CRISPR Cas9 nuclease and ribonucleoprotein may be wrapped with natural cellular membranes to provide membrane-wrapped therapeutics for drug delivery.

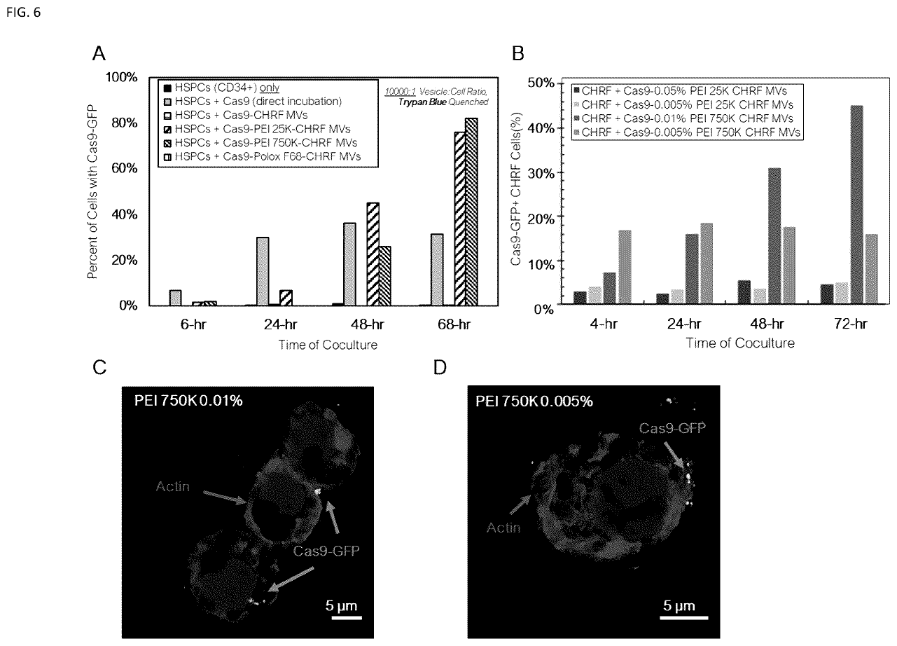

After determining our system's gene-editing effectiveness, we next created Cas9-GFP nuclease-loaded cellular membrane vesicles. For these experiments, different polymers were used to solubilize Cas9 prior to membrane encapsulation. First, positively-charged polyethyleneimine (PEI) of several different branch weights (PEI 2-kDa, PEI 25-kDa, and PEI 750-kDa) were selected due to their use in cell transfection in other studies and their ability to “neutralize” the negative zeta potential of Cas9 prior to wrapping with a negatively charged membrane. After forming a Cas9-GFP-PEI complex at dilute PEI concentrations, the Cas9 complex was successively wrapped with PKH26-stained CHRF^{PMA} membranes and incubated with both CD34+ HSPCs and CHRF cells with a protocol similar to as described in Example 4. While initial uptake of Cas9-GFP was low, the % of Cas9-GFP⁺ cells markedly increased at each successive timepoint, with over 75% of HSPCs (FIG. 6A) and nearly 50% of CHRF cells (FIG. 6B) containing Cas9-GFP after 68-72 hours of incubation with membrane-wrapped Cas9 prepared with 0.01% PEI 25-kDa or PEI 750-kDa. Furthermore, confocal microscopy confirmed these results, with Cas9-GFP visible both within and along the periphery of the CHRF cells cultured for 72 hours with Cas9-PEI CHRF cellular MVs prepared with PEI 750-kDa (FIG. 6C). The same result held after the concentration of PEI 750-kDa in the Cas9-PEI CHRF cellular MVs was halved to 0.005% (FIG. 6D), and co-localization of the Cas9-GFP and PKH26-stained membrane successfully indicate delivery of the Cas9-PEI-membrane complex to the cell. Example 1E. Cas9-loaded cellular MVs may be further purified to significantly curb the impact of PEI-induced cytotoxicity.

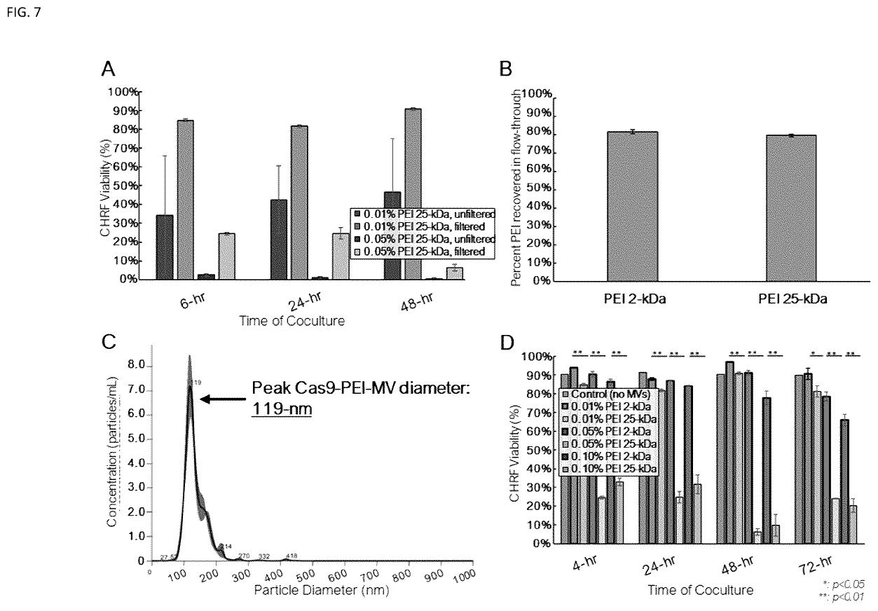

We next determined if the Cas9-PEI CHRF cellular MVs could be further purified via ultrafiltration to eliminate any cytotoxicity of “free” PEI in solution. Both unwashed and purified Cas9-PEI-25-kDa cellular MVs were subsequently incubated with CHRF cells, and the cells were screened for viability and presence of Cas9-GFP for several days via flow cytometry and microscopy. As shown in FIG. 7A, the purified ultra-filtered Cas9-PEI cellular MVs imparted significantly less cytotoxicity to CHRF cells throughout the co-incubation period. To confirm if the improved cytotoxicity was due to removal of free PEI in solution, we measured the concentration of PEI via a colorimetric assay and determined that greater than 80% of free PEI was removed (FIG. 7B) while retaining the wrapped Cas9-PEI cellular MVs (FIG. 7C). Finally, we tested the overall impact of the purification step, the PEI concentration, and branch weight of PEI on the viability of the CHRF cells, and we found that PEI 2-kDa imparted significantly less cytotoxicity across a broad range of concentrations than PEI 25-kDa (FIG. 7D); PEI 750-kDa was eliminated due to its cytotoxicity to HSPCs.

Example 1F. Cas9-loaded cellular membrane vesicles may be formulated to maximize Cas9 uptake while minimizing cytotoxicity for various cell types.

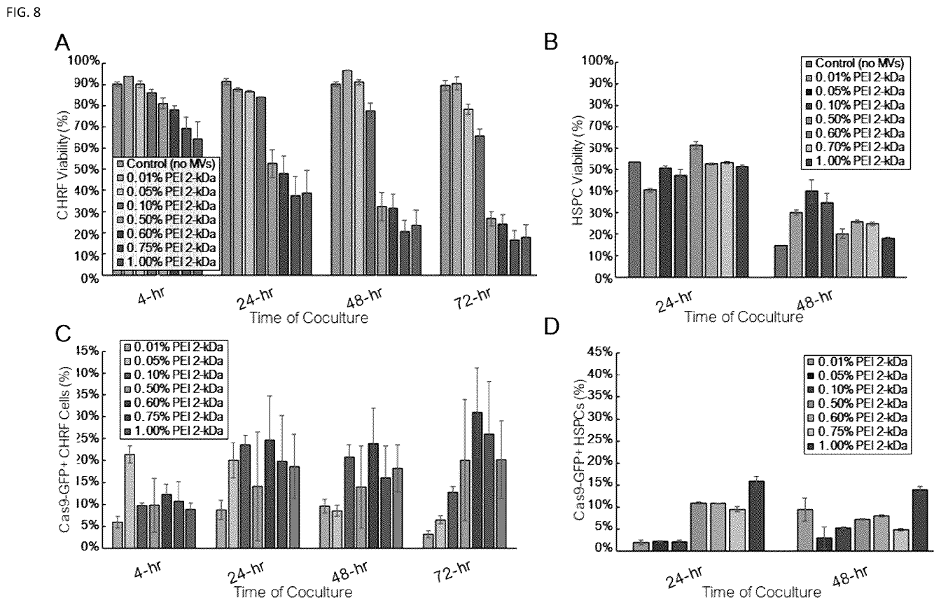

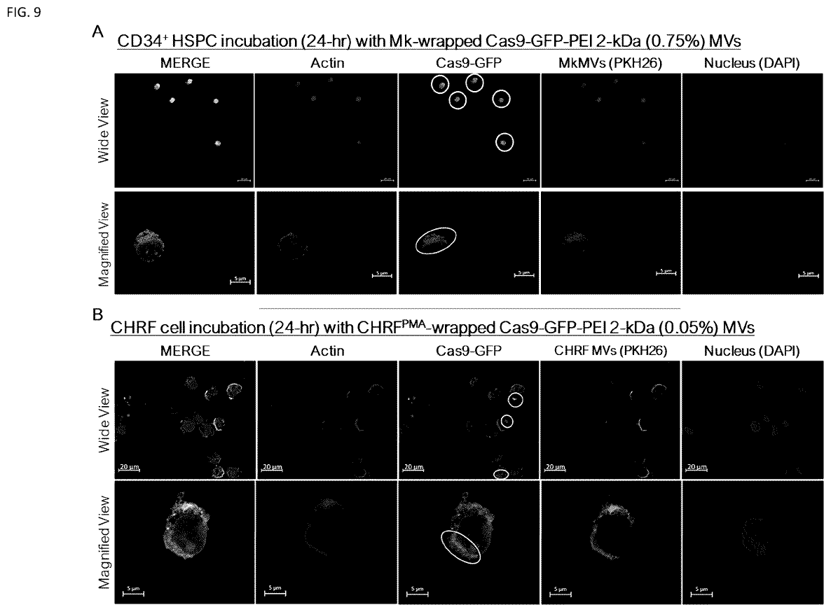

To determine the relationship between PEI concentration and Cas9 delivery efficiency, a range of PEI 2-kDa concentrations were used to prepare CHRF and Mk-wrapped Cas9-PEI cellular MVs. For this study, Cas9-PEI cellular MVs were prepared using between 0.01% to 1.00% PEI 2-kDa solutions using the procedure described in Example 1E. We first incubated the spread of Cas9-PEI CHRF cellular MVs with CHRF cells, and the cells were screened for viability and Cas9-GFP uptake over a course of 4-72 hours. We found that using concentrations of 0.10% PEI 2-kDa and below had only modest impacts on CHRF cell viability between 4- to 48-hours (FIG. 8A) with a sharp drop off in viability at higher PEI concentrations. We also found that greater than 20% of CHRF cells were Cas9-GFP fluorescent at both 4-hour and 24-hour timepoints following incubation with Cas9-PEI CHRF cellular MVs prepared 0.05% PEI 2-kDa, thus demonstrating that 0.05% PEI 2-kDa was optimal for viability and Cas9 delivery to CHRF cells. We repeated this study with HSPCs, using MkMVs in lieu of CHRF cellular MVs. While HSPCs are substantially more sensitive than CHRF cells, comparatively higher concentrations of PEI 2-kDa had less impact on HSPC viability when compared to the untreated control (FIG. 8B). Overall Cas9-GFP uptake was lower than the CHRF cells, with a maximum of 10-15% Cas9-GFP+HSPCs after 24 hours incubation with Cas9-PEI MkMVs prepared with 0.5% to 1.0% PEI 2-kDa (FIG. 8D). Thus, we were able to tune the formulation of the Cas9-PEI cellular MVs to minimize cytotoxicity and maximize Cas9 delivery to specific types of cells.

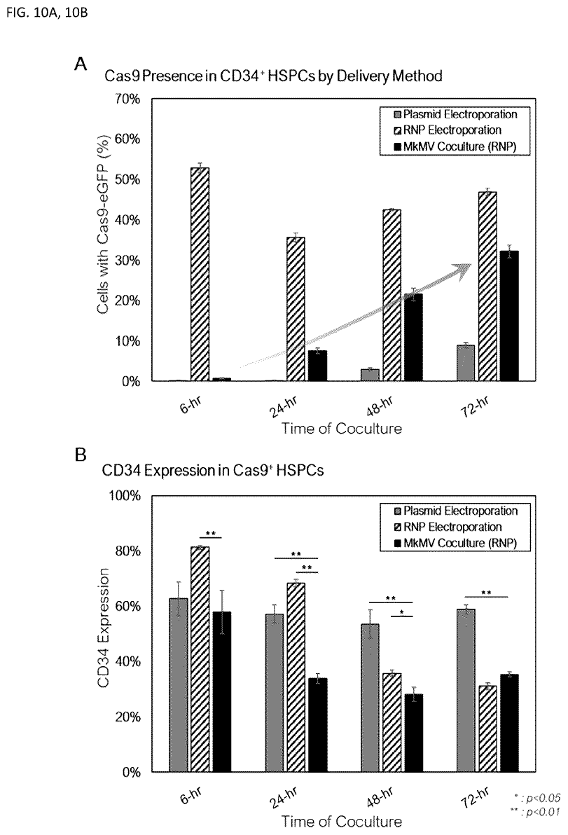

To confirm if Cas9 was successfully taken up by the cells, we used confocal microscopy to test if Cas9-GFP was within the periphery of the cell. For this study, we fixed CHRF cells and HSPCs following 24-hours incubation with Cas9-PEI (0.05%) CHRF cellular MVs and Cas9-PEI (0.75%) MkMVs, respectively. We subsequently stained the actin cytoskeleton with fluorescently-conjugated phalloidin to visualize the periphery of the cells. As the Cas9-PEI cellular MVs were prepared with PKH26-stained cell membranes, presence of Cas9-GFP and PKH26 fluorescence within the actin cytoskeleton would indicate successful Cas9 uptake. As shown in FIG. 9A, virtually all of the HSPCs in the image contained some level of GFP fluorescence, thus confirming successful Cas9-GFP uptake in HSPCs via the Cas9-PEI MkMV system. This was further validated in the magnified image, as the Cas9-GFP signal was well within the periphery of the phalloidin stained actin cytoskeleton. These phenomena were virtually similar with CHRF cells, further demonstrating the applicability of Cas9-PEI cellular MVs as a potent Cas9 delivery vehicle across a range of different cell types.

Example 1G. Cas9 ribonucleoprotein-loaded MkMVs robustly edits specific genes in HSPCs.

Finally, to test the functionality of the Cas9-PEI cellular MV system as a method for performing edits to specific genes, we prepared Cas9-PEI MkMVs with Cas9-sgRNA ribonucleoprotein (RNP). The Cas9-sgRNA RNP was assembled with gRNA complementary to the CD34 gene; CD34 is a surface marker characteristic to HSPCs. As no template DNA was being delivered alongside the Cas9-sgCD34 RNP, Cas9-induced double-stranded breaks within the CD34 gene would yield CD34 gene disruption via formation of indels following DNA self-repair. Thus, successful Cas9 gene editing could be determined through a reduction of CD34 expression in HSPCs following incubation with Cas9-sgCD34-PEI MkMVs.

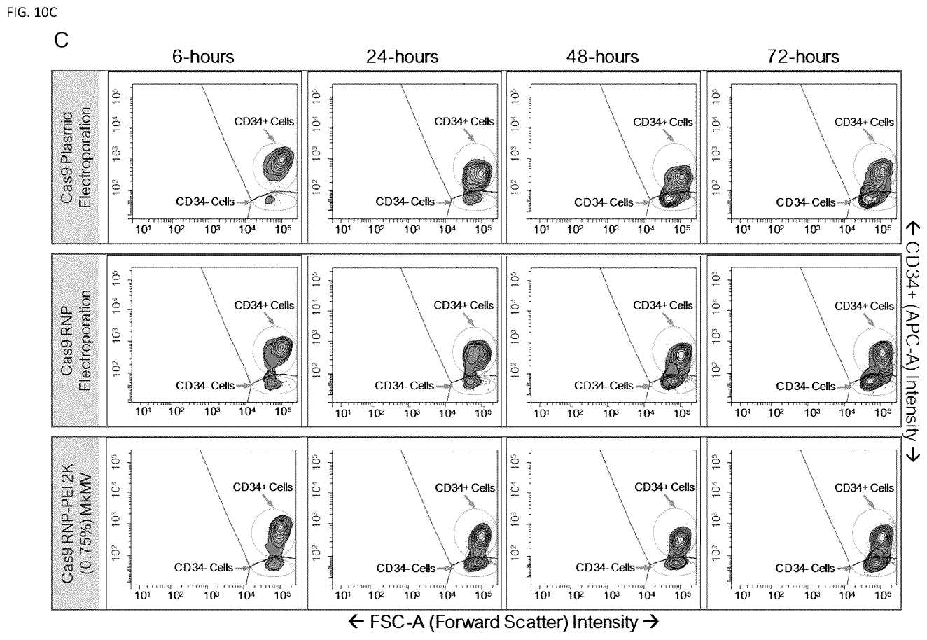

For the study, HSPCs were either incubated with the Cas9-GFP-sgCD34-PEI MkMVs or were electroporated with Cas9-GFP-sgCD34 expressing plasmid DNA or Cas9-GFP-sgCD34 RNPs. As expected, direct electroporation of the Cas9-sgCD34 RNP provided the greatest proportion of Cas9-GFP fluorescent HSPCs across all timepoints (FIG. 10A), while incubation with the Cas9-sgCD34 PEI MkMVs resulted in more gradual uptake of the RNP. However, the Cas9-sgCD34 PEI MkMVs were highly effective at disrupting CD34 expression, especially after 24 hours; less than 40% of Cas9-GFP+ HSPCs incubated with the Cas9-sgCD34 PEI MkMVs expressed CD34 compared to greater than 65% for both plasmid DNA and RNP-electroporated HSPCs (FIG. 10B). This shows that Cas9 may be more functional intracellularly when delivered via the Cas9-PEI cellular MV system rather than direct transfection. The gradual uptake of Cas9 into HSPCs over time could also have benefits, as the Cas9-PEI cellular MV system could facilitate more controlled Cas9 delivery and subsequent Cas9-mediated gene therapy.

Example 1H. Cas9-loaded CHRF and MkMVs combine efficiency of Cas9 nucleoprotein gene editing with cell-specific delivery for targeted gene therapy.

Following successful production of Cas9-loaded cellular MVs, our system can provide precise gene edits of CHRF cells via co-incubation of CHRF cell cultures with Cas9+gRNA-loaded cellular MVs. As the cellular MVs share many of the same characteristics as naturally-produced microparticles and exosomes which are linked to cell-to-cell communication and biomolecule transport, the Cas9-laden cellular MVs will efficiently deliver Cas9 to the target cells. The editing efficiency may be easily screened through disruption of a surface marker, as explained in Examples 1 and 2. To demonstrate the clinical relevance of gene silencing based therapy, transient knockdown of the BCL11A gene, which is responsible for γ-globin repression, has been shown to correct sickle cell disease (SCD). While epigenetic means has been used to silence BCL11A, our Cas9 MV cellular MVs may provide a longer-term remedy by knockout of the gene via indel formation.

Using the Cas9-loaded cellular membrane vesicles as a platform for delivering Cas9 and Cas9-related cargo may also be applied to gene repair. As we demonstrate with our Cas9+sgRNA plasmid cellular MVs, cellular membranes may encapsulate a combination of the Cas9-sgRNA nucleoprotein with a short donor template. Conveniently, designing the Cas9 cellular MVs for gene replacement does not require any fundamental changes to the cellular MV loading process, as the gene segment for knock-in may be co-delivered alongside the Cas9 nuclease, nucleoprotein, and/or the sgRNA-expressing plasmid. Thus, the versatility of the Cas9 MkMV system allows it to be used for a broad spectrum of gene therapies.

Example 1I. Cas9-loaded MkMVs facilitate gene repair in HSPCs, which ultimately generates blood cells with corrected phenotype.

Using megakaryocytic or megakaryocyte-like cellular membrane vesicle for Cas9 extends direct, cell-specific gene editing to HSPCs. As described earlier, in our presented system, natural moieties found on the surface of the outer membrane vesicle (receptors, ligands etc.) facilitate homotypic binding with target cells without the need for additional protein engineering. This greatly improves both the efficiency and impact of Cas9-mediated gene therapy of blood diseases. As HSPCs may differentiate into different lineages of blood cells, any repairs done to remediate faulty genes within the HSPCs will likely yield “corrected” blood cells. The designed Cas9 MkMVs may facilitate HSPC-specific gene repair and corrected myeloid, lymphoid, and erythrocyte progenitors, thus multiplicatively producing various blood cells with a healthy phenotype (FIG. 11). Ultimately, a much smaller therapeutic dose of the Cas9 MkMVs will be required to remediate any genetic blood disease, therefore making the HSPC-targeting Cas9 MkMVs a highly potent therapeutic.

Example 1J. Cas9 cellular MVs may be administered intravenously for in vivo gene therapy of various hematological disorders.

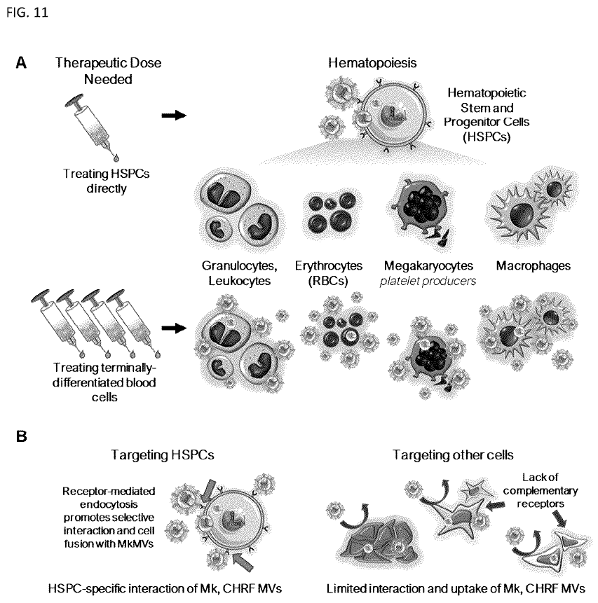

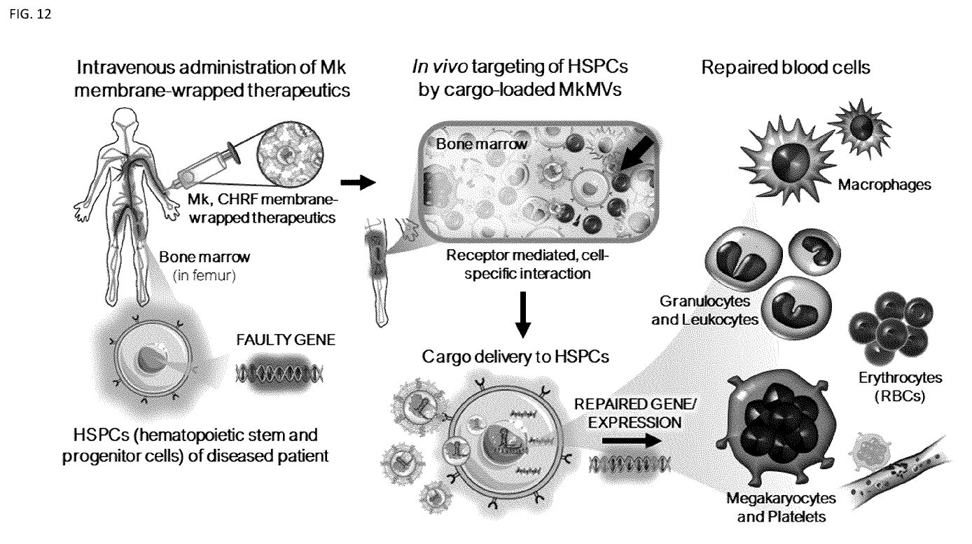

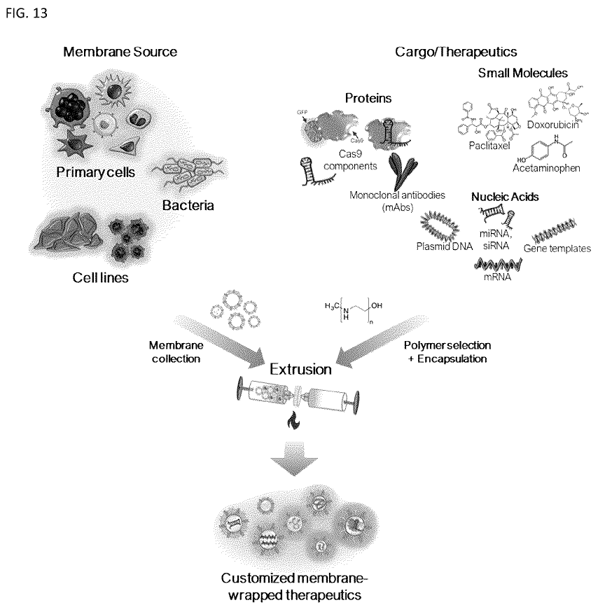

After confirming effective targeted Cas9-mediated gene therapy of HSPCs in vitro, we expect to observe similar results in vivo. One of the purported benefits of Cas9 and other cargo-loaded MkMVs is its ability to facilitate in vivo gene therapies for clinical treatment of a variety of hematological genetic diseases. Current methods for addressing these genetic maladies rely extensively on either ex vivo gene therapy of HSPCs or transient epigenetic therapy via RNA interference and other means. Many previous arts involving homotypic targeting require specific engineering of receptors on the surface of the delivery vehicle to facilitate complementary binding to the target cell or tissue; in our system these modifications and the need for any protein engineering is bypassed by the natural function and HSPC-targeting behavior of the MkMVs and CHRF cellular MVs (FIG. 12). This may greatly: 1) increase the bioavailability of any delivered Cas9 and other therapeutics, 2) simplifies gene therapy through direct delivery to the patient, and 3) provides optimal customizability for each patient's individual gene therapy needs.

Example 1K. Cellular membrane vesicles like the Mk-membrane vesicles of the examples above may be also generated by membranes of other cells for targeted delivery to other cell types based on the receptors on the membranes and their ability to recognize specific cell targets.

Membranes from other blood-type cells, such as granulocytic, erythroid, monocytic cells, macrophages and lymphocytic cells (T cells and B cells) are used to make cellular membrane vesicles and load them as in the examples above with various cargo molecules for homotypic delivery to cognate target cells. Similarly, other stem cells, endothelial cells, cardiac cells, bone cells, and neuronal cells are used to make cellular membrane vesicles and load them as in the examples above with various cargo molecules for homotypic delivery to cognate target cells.

### DISCUSSION

All prior work for delivering cargo to cells in vitro or in vivo using biological membranes used either wrapped solid or semisolid nanoparticles or natively produced EVs that carry a lot of cellular material from the parent cell. Our inventions use purified membranes from cells without containing either a synthetic nanoparticle or native cellular material. We wrap proteins and other cargo in a liquid state with select membranes that have targeting capability. There is no native cargo in our cellular membrane vesicles. Synthetic solid or semisolid nanoparticles loaded with cargo and wrapped with biological membranes have been previously disclosed as summarized below. EVs from mammalian cells or bacterial cells have also been used for delivery, but those contain a lot of native cellular cargo from the parent cells, and may thus induce undesirable effects to the target cells. The composition of our cellular membrane vesicles is very different from the composition of either native EVs or of membrane-wrapped nanoparticles.

The versatility and efficiency of CRISPR Cas9-based gene therapy improves with simultaneous delivery of decoupled Cas9 nuclease and sgRNA. Cas9-mediated gene therapy has already showcased its potential in treating genetic disorder, including ubiquitous blood disorders such as sickle cell anemia and certain myeloid leukemias. As we show in Examples 1A, 1B, and 1C, delivering Cas9 as a preformed, fully-synthesized nuclease greatly improves the gene editing efficiency over solely plasmid-based approaches. These results reflect similar approaches taken by other scientists in addressing possible shortfalls in Cas9-mediating gene editing. For example, an upwards of 70% downregulation of the Plk1 oncogene has been demonstrated using a Cas9 nuclease system, therefore showing the high efficiency of the nuclease-based system.

Here we demonstrate the ability for the various components to be “mixed and matched”, largely stemming from decoupling. First, Cas9-based therapeutics may be scaled, as Cas9-loaded cellular MVs may be mass-produced as Cas9 is nonspecific to any gene without the gene-complementing sgRNA; cell specificity may be imparted later via wrapping with different cellular membranes. Next, different sgRNAs with different targets, either within the same gene or totally disparate genes, may be administered by simply loading the specific plasmids expressing for each sgRNA into discrete cellular MVs. This may allow simultaneous Cas9-mediated treatments for different gene therapies. Finally, the dose response from a patient is tunable, as the efficiency of the Cas9-mediated therapy may be revised by increasing or decreasing delivery of Cas9-loaded and/or sgRNA-loaded cellular MVs.

Cas9, nucleic acid, and other cargo alone or with liquid polymers may be wrapped in cellular membranes for cargo delivery to specific cells. To facilitate efficient delivery of the Cas9 nuclease to cells, it must first be encased in an engineered cellular membrane vesicle to withstand clearance from macrophages and permit membrane-wrapping for homotypic delivery to different cells. We have demonstrated this concept with purified Cas9-GFP, and we have found that Cas9-GFP solution may be effectively electrostatically coated with polyethyleneimine (PEI), a positively-charged branched liquid polymer, which may be further wrapped with natural cell membranes. Due to the positive charge of PEI and the negative charges of both Cas9 and the cellular membrane vesicles, PEI permits effective, electrostatically-favorable membrane wrapping of Cas9 and other negatively-charged cargo, thus creating a semi-synthetic and cell-specific vessel for cargo delivery.

Positively-charged polymers such as polyethyleneimine are also instrumental in facilitating cargo delivery into the cell due the polymer's propensity to disrupt both oppositely-charged outer cell membranes and intracellular lysosomes. By combining the properties of PEI and other cargo-compatible polymers with the targeting ability of naturally-derived cell membrane vesicles, we demonstrate the potential to develop a highly tunable cargo nanocarrier and delivery vehicle. This property also allows the formulation of the Cas9-PEI cellular MVs to be tuned based on the cell's compatibility with PEI, thus facilitating optimal Cas9 delivery to specific types of cells e.g., HSPCs, Mk-like cells etc.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A stable non-naturally occurring cellular membrane vesicle for delivering an active agent into a target cell, comprising a biological membrane from a parent cell and a liquid medium encapsulated by the biological membrane, wherein the liquid medium comprises an active agent and does not comprise a native cytoplasmic component of the parent cell, wherein the biological membrane is specific for the target cell, and wherein the active agent remains active upon delivery into the target cell.

2. The stable non-naturally occurring cellular membrane vesicle of claim 1, wherein the cellular membrane vesicle consists of the biological membrane and the liquid medium, and wherein the cellular membrane vesicle has a diameter of 100-1000 nm.

3. (canceled)

4. The stable non-naturally occurring cellular membrane vesicle of claim 1, wherein the biological membrane comprises a native surface receptor of the parent cell, and the native surface receptor binds specifically to the target cell, wherein the parent cell is selected from the group consisting of megakaryocytes (Mks), granulocytes, erythrocytes, platelets, monocytes, macrophages, lymphocytes, stem cells, endothelial cells, cardiac cells, bone cells, neuronal cells and tumor cells, and wherein the target cell is selected from the group consisting of hematopoietic stem and progenitor cells (HSPCs), adult stem cells, cardiac cells and neuronal cells.

5. (canceled)

6. (canceled)

7. The stable non-naturally occurring cellular membrane vesicle of claim 1, wherein the parent cell is a megakaryocyte (Mk) and the target cell is a hematopoietic stem and progenitor cell (HSPC).

8. The stable non-naturally occurring cellular membrane vesicle of claim 1, wherein the active agent is selected from the group consisting of proteins, nucleoproteins, nucleic acids, organic molecules, and combinations thereof.

9. The stable non-naturally occurring cellular membrane vesicle of claim 1, wherein the target cell expresses a guide RNA (gRNA) specific for a target native gene of the target cell, and the active agent comprises Cas9 that binds specifically to the gRNA, or the active agent comprises a ribonucleoprotein (RNP) of Cas9 and a guide RNA (gRNA) specific for a target native gene of the target cell.

10. (canceled)

11. The stable non-naturally occurring cellular membrane vesicle of claim 9, wherein the active agent further comprises a therapeutic.

12. The stable non-naturally occurring cellular membrane vesicle of claim 1, wherein the liquid medium further comprises a soluble polymer selected from the group consisting of polycation polyethyleneimine (PEI), non-toxic polycations, polyanionic polymers, and nonionic triblock copolymers, optionally wherein the soluble polymer is PEI.

13. (canceled)

14. (canceled)

15. A method for delivery of an active agent into a target cell, comprising:
(a) contacting the stable non-naturally occurring cellular membrane vesicle of claim 1 with the target cell, and
(b) releasing the active agent into the target cell from the cellular membrane vesicle, wherein the active agent remains active upon release into the target cell.

16. The method of claim 15, wherein the active agent is released into the target cell within 120 minutes after the contacting step.

17. The method of claim 15, further comprising fusing the biological membrane with a cytoplasmic membrane of the target cell after the contacting step and before the releasing step.

18. The method of claim 15, wherein the target cell expresses a guide RNA (gRNA) specific for a target native gene of the target cell, and the active agent comprises Cas9 that binds specifically to the gRNA, or the active agent comprises a ribonucleoprotein (RNP) of Cas9 and a guide RNA (gRNA) specific for a target native gene of the target cell.

19. (canceled)

20. The method of claim 18, further comprising editing of the target native gene in the target cell.

21. The method of claim 15, wherein the parent cell is a megakaryocyte (Mk), the target cell is a hematopoietic stem and progenitor cell (HSPC), and the active agent comprises a ribonucleoprotein (RNP) of Cas9 and a guide RNA (gRNA) specific for a target native gene in the HSPC, or wherein the parent cell is a megakaryocyte (Mk), the target cell is a hematopoietic stem and progenitor cell (HSPC) expressing a guide RNA (gRNA) specific for a target native gene of the target cell, and the active agent comprises Cas9 that binds specifically to the gRNA.

22. The method of claim 21, further comprising editing the target native gene in the target cell.

23. (canceled)

24. (canceled)

25. The method of claim 15, wherein the liquid medium further comprises a soluble polymer selected from the group consisting of polycation polyethyleneimine (PEI), non-toxic polycations, polyanionic polymers, and nonionic triblock copolymers.

26. (canceled)

27. (canceled)

28. A method of preparing the stable non-naturally occurring cellular membrane vesicle of claim 1, comprising
(a) isolating the biological membrane from the parent cell, and
(b) encapsulating the liquid medium by the biological membrane, wherein the liquid medium comprises the active agent, whereby the stable non-naturally occurring cellular membrane vesicle is prepared.

29. The method of claim 28, further comprising mixing the isolated biological membrane and the biological liquid medium, wherein the liquid medium further comprises a soluble polymer selected from the group consisting of polycation polyethyleneimine (PEI), non-toxic polycations, polyanionic polymers, and nonionic triblock copolymers, whereby a mixture is obtained, and extruding the mixture through an extruder pore.

30. (canceled)

31. (canceled)

32. The method of claim 29, wherein the isolated biological membrane and the biological liquid medium are mixed at a weight ratio from 10:1 to 1:10.

33. The method of claim 29, wherein the extruder pore has a diameter from 1 nm to 1000 nm.
